(19) United States
(12) Patent Application Publication
(10) Pub. No.: US 2022/0259235 A1
(43) Pub. Date: Aug. 18, 2022

(54) EGFR Inhibitor, Composition, and Preparation Method Therefor

(71) Applicant: BETTA PHARMACEUTICALS CO., LTD, Hangzhou, Zhejiang (CN)

(72) Inventors: Xiangyong Liu, Beijing (CN); Changyong Qiu, Beijing (CN); Qichao Shen, Beijing (CN); Mengqiang Liu, Beijing (CN); Haitong Sheng, Beijing (CN); Xiaodong Song, Beijing (CN); Guolong Du, Beijing (CN); Jiabing Wang, Beijing (CN); Lieming Ding, Hangzhou, Zhejiang (CN)

(21) Appl. No.: 17/629,976

(22) PCT Filed: Jul. 23, 2020
(86) PCT No.: PCT/CN2020/103884

§ 371 Date: Jan. 25, 2022

(30) Foreign Application Priority Data
Jul. 26, 2019 (CN) 201910686970.4

(51) Int. Cl. C07F 9/6558; A61P 35/00; C07F 9/6561

(52) U.S. Cl. CPC C07F 9/65583; C07F 9/6561; A61P 35/00

(57) ABSTRACT

The present invention relates to compounds of Formula I, methods of using the compounds as EGFR inhibitors, and pharmaceutical compositions comprising such compounds. The compounds are useful in treating, preventing or ameliorating diseases or disorders such as cancer or infections.

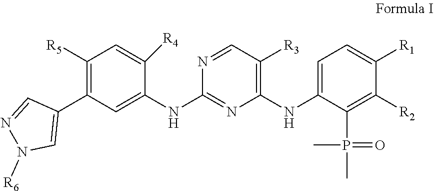

### FIELD OF THE INVENTION

The invention is concerned with pharmaceutically active compounds, deuterated compounds (hydrogen substituted with deuterium) and pharmaceutically acceptable salts thereof, which can be used to treat or prevent diseases or medical conditions mediated by certain mutant forms of epidermal growth factor receptor (For example, L858R activating mutant, Exon19 deletion activating mutant, T790M resistant mutant and C797S resistant mutant). The invention also relates to a pharmaceutical composition comprising the compound, and a method of using the compound, deuterated compound and their relating salts to treat diseases mediated by various forms of EGFR mutants.

### BACKGROUND OF THE INVENTION

Epidermal growth factor receptor (EGFR) is a kind of transmembrane glycoprotein belonging to the ErbB family of tyrosine kinase receptors. Activation of EGFR leads to autophosphorylation of receptor tyrosine kinases, which are involved in the cascade of downstream signal transduction pathway that regulate cell proliferation, differentiation, and survival. EGFR is abnormally activated by various mechanisms, such as receptor overexpression, mutation, ligand-dependent receptor dimerization, ligand-independent activation, and is related to the development of a variety of human cancers.

EGFR inhibition is one of the key targets of cancer therapy. Despite the rapid development of previous generations of EGFR-Tkis, the problem of drug resistance has also emerged with the development of drugs. Most drug resistance is due to the T790M mutation of the ATP receptor. Recently developed third-generation irreversible inhibitors for T790M, such as osimertinib, have good activity on inhibitory, but drug resistance will inevitably appear. Most drug resistance is due to the T790M mutation of the ATP receptor. Recently developed third-generation irreversible inhibitors for T790M, such as osimertinib, have good activity on inhibitory, but drug resistance will inevitably appear. C797S mutation is a kind of missense mutation in which serine replaces cysteine at site 797 of exon 20 of EGFR is located in the tyrosine kinase region of EGFR. C797S mutation prevents osimertinib from forming covalent bonds in the ATP binding domain, thus losing its inhibitory effect on EGFR activation and resulting in drug resistance.

Early patent applications WO2018108064, WO2018115218, WO2018181777 disclosed a series of fourth-generation EGFR inhibitors, but there is still a need for EGFR C797S inhibitors with stronger activity and better PK. In the invention, the applicant discovers a kind of small molecules that can be used as the fourth-generation EGFR inhibitor, whose activity can be used to treat cancer and/or infectious diseases. These small molecules are expected to be used as drugs with better stability, solubility, bioavailability, therapeutic indices and toxicity values, which are essential to develop effective drugs for human health.

### SUMMARY OF INVENTION

The present invention relates to compounds capable of inhibiting EGFR which can be useful in the treatment of cancers and infectious diseases.

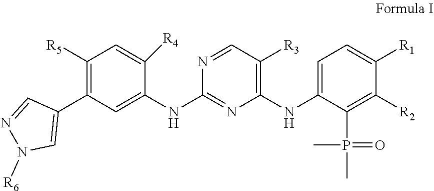

Wherein,

R₁ and R₂ are independently selected from halogens, CN, NH₂, —C₁₋₆ alkyl, —C₁₋₆ alkoxy and —C₃₋₅ cycloalkyl, respectively; the NH₂, —C₁₋₆ alkyl, —C₁₋₆ alkoxy and —C₃₋₅ cycloalkyl are optionally substituted with halogens and —C₁₋₄ alkyl; or

R₁ and R₂ together with the atoms to which they are attached form phenyl, —C₅₋₆ heteroaryl, —C₅₋₇ heterocyclyl or —C₅₋₆ cycloalkyl; the phenyl, —C₅₋₆ heteroaryl, —C₅₋₇ heterocyclyl or —C₅₋₆ cycloalkyl are optionally substituted with halogen, CN, NH2, —C₁₋₆ alkyl;

R₃ is selected from hydrogen, halogen and —C₁₋₆ alkyl;

R₄ is selected from hydrogen, halogen, —C₁₋₆ alkyl, —C₁₋₆ alkoxy and —C₁₋₆

R₅ is selected from —C₅₋₆ heterocyclyl,

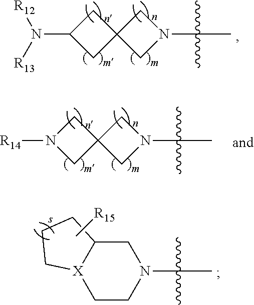

the —C₅₋₆ heterocyclyl is optionally substituted with —C₄₋₆ cycloalkyl, —C₄₋₆ heterocyclyl and —NR₇R₈;

R₆, R₇, R₅, R₁₂, R₁₃, R₁₄ and R₁₅ are independently selected from hydrogen, —C₁₋₆ alkyl and —C₁₋₆ halogenated alkyl, respectively;

X is selected from CH and N;

m, n, m′, n′, and s are independently selected from 1 and 2, respectively.

The embodiment compound of Formula I, wherein R₁ and R₂ are independently selected from —C₁₋₆ alkyl and —C₃₋₅ cycloalkyl, respectively.

The embodiment compound of Formula I, wherein R₁ and R₂ are independently selected from —CH₃ and

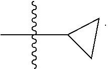 [SMILES: *C1CC1]

The embodiment compound of Formula I, wherein R₁ and R₂, together with the atoms to which they are attached form —C₅₋₆ heteroaryl; the —C₅₋₆ heteroaryl is optionally substituted with hydrogen, halogen, CN, NH₂, —C₁₋₃ alkyl, and —C₃₋₅ cycloalkyl.

The embodiment compound of Formula I, wherein R₁ and R₂ together with the atoms to which they are attached form

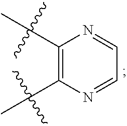 [SMILES: *c1nccnc1*]

The embodiment compound of Formula I, wherein R₁ and R₂ together with the atoms to which they are attached form

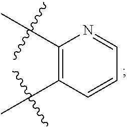 [SMILES: *c1cccnc1*]

The embodiment compound of Formula I, wherein R₃ is selected from halogen;

The embodiment compound of Formula I, wherein R₃ is selected from Cl and Br;

The embodiment compound of Formula I, wherein R₃ is selected from hydrogen;

The embodiment compound of Formula I, wherein R₄ is selected from —C₁₋₆ alkoxy;

The embodiment compound of Formula I, wherein R₄ is selected from —O—CH₃;

The embodiment compound of Formula I, wherein R₅ is selected from —C₅₋₆ heterocyclyl;

The embodiment compound of Formula I, wherein R₅ is selected from

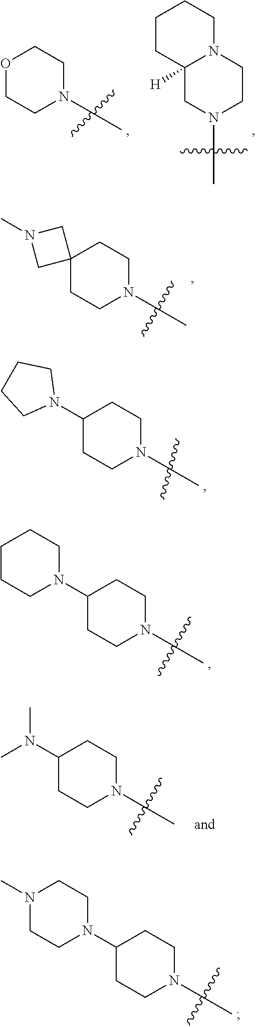

The embodiment compound of Formula I, wherein R₆ is selected from hydrogen, —C₁₋₃ alkyl and halogenated alkyl;

The embodiment compound of Formula I, wherein R₆ is selected from H, CH₃, CH₂CH₃ and CHF₂.

The embodiment compound of Formula I, wherein:
1) (6-((5-Chloro-2-((2-methoxy-5-(1-methyl-1hydro-pyrazol-4-yl)-4-(4-(4-methylpiperazine-1-yl) piperidin-1-yl)phenyl)amino)pyrimidin-4-yl)amino)quinoxalin-5-yl)dimethylphosphine oxide;
2) (6-((5-Chloro-2-((4-(4-(dimethylamino)piperidin-1-yl)-2-methoxy-5-(1-methyl-1H-pyrazole-4-yl) phenyl)amino)pyrimidin-4-yl)amino)quinoxalin-5-yl)dimethylphosphine oxide;
3) (6-((5-Chloro-2-((4-(4-(dimethylamino)piperidin-1-yl)-2-methoxy-5-(1H-pyrazol-4-yl)benzene)amino)pyrimidin-4-yl) amino)quinoxalin-5-yl)dimethylphosphine oxide;
4) (6-((5-Chloro-2((4-(4-(dimethylamino)piperidin-1-yl)-2-methoxy-5-(1-methyl-1H-pyrazole-4-yl)phenyl)amino)pyrimidin-4-yl) amino)-2,3-dimethylphenyl)dimethylphosphine oxide;
5) (6-((2-((4-([1,4′-Dipiperidine]-1′-yl)-2-methoxy-5-(1-methyl-1H-pyrazole-4-yl)Phenyl)amino)-5-chloropyrimidin-4-yl) amino)quinoxalin-5-yl)dimethylphosphine oxide;
6) (6-((5-Chloro-2-((2-methoxy-5-(1-methyl-1H-pyrazol-4-yl)-4-(4-(pyrrol-1-yl) piperidine-1-yl)phenyl)amino)pyrimidin-4-yl)yl)-2,3-dimethylphenyl)dimethylphosphine oxide;
7) (6-(5-chloro-2-((2-methoxy-5-(1-methyl-1H-pyrazol-4-yl)-4-4-(2-methyl-2,7-diazaspiro[3.5]nonyl-7-yl)phenyl)amino)pyrimidin-4-yl)amino)-2,3-dimethylphenyl)dimethylphosphine oxide;
8) (R)-(6-((5-chloro-2-((2-methoxy-5-methyl-1H-pyrazol-4-yl)-4-(octahydro-2hydro-Pyridine [1,2-a]pyrazine-2-yl)phenyl)amino)pyrimidin-4-yl)amino)-2,3-dimethylphenyl)dimethylphosphine oxide;
9) (6-((5-chloro-2-(2-methoxy-5-(1-methyl-1hydro-pyrazole)-4-morpholinephenyl) amino)pyrimidine-4-amino)quinoxalin-5-dimethylphosphine oxide;
10) (6-((5-Bromo-2-((2-methoxy-5-(1-methyl-1H-pyrazol-4-yl)-4-(4-(4-methylpiperazine-1-yl) piperidin-1-yl)phenyl)amino)pyrimidin-4-yl)amino)quinoxalin-5-yl)dimethylphosphine oxide;
11) (6-((2((4-([1,4′-Bipiperidine]-1′-yl)-2-methoxy-5-(1-methyl-1H-pyrazole-4-yl) phenyl)amino)-5-bromopyrimidine-4-yl)amino)quinoxalin-5-yl)dimethylphosphine oxide;
12) (6-((5-Bromo-2-((2-methoxy-5-(1-methyl-1H-pyrazol-4-yl)-4-(4-(4-methylpiperazine-1-yl) piperidin-1-yl)phenyl)amino)pyrimidin-4-yl)amino)-3-cyclopropyl-2-methyl)dimethylphosphine oxide;
13) (6-((2-((4-([1,4′-Bipiperidine]-1′-yl)-2-methoxy-5-(1-methyl-1H-pyrazole-4-yl) phenyl)amino)-5-bromopyrimidine-4-yl)amino)-3-cyclopropyl-2-methylphenyl)dimethylphosphine oxide;
14) (6-((5-Bromo-2-((2-methoxy-5-(1-methyl-1h-pyrazol-4-yl)-4-(4-(4-methylpiperazine-1-yl) piperidin-1-yl)phenyl)amino)pyrimidin-4-yl)amino)-2,3-dimethylphenyl)dimethylphosphine oxide;
15) (6-((2-((4-([1,4′-Bipiperidine]-1′-yl)-2-methoxy-5-(1-methyl-1H-pyrazole-4-yl) phenyl)amino)-5-bromopyrimidin-4-yl)amino)-2,3-dimethylphenyl)dimethylphosphine oxide;
16) (6-((2-((4-([1,4′-Bipiperidine]-1′-yl)-2-methoxy-5-(1-methyl-1H-pyrazole-4-yl)phenyl)amino)-5-bromopyrimidine-4-yl)amino)-3- cyclopropyl-2-methylphenyl)dimethylphosphine oxide;
17) (6-((5-Bromo-2-((2-methoxy-5-(1-methyl-1h-pyrazol-4-yl)-4-(4-(4-methylpiperazine-1-yl) piperidin-1-yl)phenyl)amino)pyrimidin-4-yl)amino)-2,3-dimethylphenyl)dimethylphosphine oxide;
18) (R)-(6-((5-Chloro-2-((2-methoxy-5-([1-methyl-1hydro-pyrazol-4-yl]-4-(octahydro-2hydro-pyrazol[1,2-a]pyrazine-2-yl) phenyl)amino)pyrimidin-4-yl)amino)quinoxalin-5-yl)dimethylphosphine oxide;
19) (6-((5-Bromo-2-((5-(1-ethyl-1H-pyrazol-4-yl)-2-methoxy)-4-(4-(4-methylpiperazine-1-yl) piperidin-1-yl)phenyl)amino)pyrimidin-4-yl)amino)quinoxalin-5-yl)dimethylphosphine oxide;
20) (6-((2-((4-([1,4′-Bipiperidine]-1′-yl)-5-(1-ethyl-1H-pyrazol-4-yl)-2-methoxyphenyl) amino)-5-bromopyrimidine-4-yl)amino)-2,3-dimethylphenyl)dimethylphosphine oxide;
21) (6-((5-Chloro-2-((5-(1-methyl-1hydro-pyrazol-4-yl)-2-methoxy-4-morpholinophenyl) amino)pyrimidine-4-Amino)2,3-dimethylphenyl)-dimethylphosphine oxide;
22) (6-((5-Bromo-2-((5-(1-ethyl-1h-pyrazol-4-yl)-2-methoxy-4-(4-(4-methylpiperazine-1-yl) piperidin-1-yl)phenyl)amino)pyrimidine-4-yl)amino)-2,3-dimethylphenyl)dimethylphosphine oxide;
23) (6-(2-(5-((1-Ethyl-1hydro-pyrazol-4-yl)-2-methoxy-4-morpholinophenyl) amino)pyrimidine-4-amino)-2,3 dimethylphenyl)dimethylphosphine oxide;
24) (6-((5-Bromo-2-((5-(1-ethyl-1H-pyrazol-4-yl)-2-methoxy-4-morpholinophenyl) amino)pyrimidine-4-Amino)-2-cyclopropylquinolin-5-yl)dimethylphosphine oxide;
25) (6-((5-Bromo-2-((4-(4-cyclopentylpiperazine-1-yl)-2-methoxy-5-(1-methyl-1H-pyrazole-4-yl) phenyl)amino)pyrimidin-4-yl)amino)quinoxalin-5-yl) dimethylphosphine oxide;
26) (6-((5-Bromo-2-((4-(4-cyclopentylpiperazine-1-yl)-2-methoxy-5-(1-methyl-1H-pyrazole-4-yl) phenyl)amino)pyrimidin-4-yl)amino)-2,3-dimethylphenyl)dimethylphosphine oxide;
27) (6-((5-Bromo-2-((4-(4-cyclopentylpiperazine-1-yl)-2-methoxy-5-(1-methyl-1H-pyrazole-4-Yl) phenyl)amino)pyrimidin-4-yl)amino)-2-cyclopropylquinolin-5-yl)dimethylphosphine oxide; or
28) (6-((5-Chloro-2-((2-methoxy-5-(1-methyl-1H-pyrazol-4-yl)-4-(4-(4-methylpiperazine-1-yl) piperidine-1-yl)phenyl)amino)pyrimidin-4-yl)amino)-2-cyclopropylquinolin-5-yl)dimethylphosphine, oxide).

The compound of Formula I, or its stereoisomer, tautomer, deuterated compound, pharmaceutically acceptable salt, prodrug, chelate, non-covalent complex or its solvate;

The present invention further provides a pharmaceutical composition, including a compound of any one of the present invention, or a pharmaceutically acceptable salt or a stereoisomer thereof, and at least one pharmaceutically acceptable carrier or excipient.

The present invention further provides methods for inhibiting various forms of EGFR, including L858R, Δ19del, T790M and C797S, said method comprising administering to a patient any of the compounds, pharmaceutically acceptable salts or a stereoisomer thereof.

The present invention also provides methods for treating EGFR-driven cancer, said method comprising administering to a patient in need thereof a therapeutically effective amount of any of the compounds or pharmaceutically acceptable salt or stereoisomer thereof.

In some implementations, the EGFR-driven cancer is characterized by the presence of one or more mutations from: (i) C797S, (ii) L858R and C797S, (iii) C797S and T790M, (iv) L858R, T790M, and C797S, or (v) Δ19del, T790M and C797S.

In some implementations, the EGFR-driven cancers involve colon cancer, stomach cancer, thyroid cancer, lung cancer, leukemia, pancreatic cancer, melanoma, brain cancer, kidney cancer, prostate cancer, ovarian cancer, or breast cancer.

In some implementations, the mentioned lung cancer is non-small cell lung cancerr caused by EGFR^{L858R/T790M/C797S} or EGFR^{Δ19del/T790M/C797S} mutant.

The present invention provides a method for inhibiting mutant EGFR in a patient, including the administration of a therapeutically effective amount of any of the compounds or its pharmaceutically acceptable salt or stereoisomer from the present invention to patients requiring such treatment.

The present invention also provides the use of the compound of the present invention or its pharmaceutical composition in the preparation of medicines.

In some implementations, wherein the mentioned drugs are used to treat or prevent cancer.

In some implementations, wherein the cancers involve colon cancer, stomach cancer, thyroid cancer, lung cancer, leukemia, pancreatic cancer, melanoma, brain cancer, kidney cancer, prostate cancer, ovarian cancer, or breast cancer.

In some implementations, the mentioned lung cancer is non-small cell lung cancerr caused by EGFRL^{858R/T790M/C797S} or EGFR^{Δ19del/T790M/C797S} mutant.

The general chemical terms used in the above general formula have their usual meanings. For example, the term “halogen” used herein refers to fluorine, chlorine, bromine or iodine unless otherwise specified. Preferred halogen groups include F, Cl and Br.

Unless otherwise stated, the alkyl used herein includes saturated monovalent hydrocarbonyl with straight-chain, branched or cyclic moieties. For example, alkyl includes methyl, ethyl, propyl, isopropyl, cyclopropyl, n-butyl, isobutyl, sec-butyl, tort-butyl, cyclobutyl, n-pentyl, 3-(2-Methyl) butyl, 2-pentyl, 2-methylbutyl, neopentyl, cyclopentyl, n-hexyl, 2-hexyl, 2-methylamyl and cyclohexyl. Similarly, C₁₋₈ in C₁₋₈ alkyl is defined as identifying the group with a straight-chain or branched arrangement with 1, 2, 3, 4, 5, 6, 7 or 8 carbon atoms.

The alkoxy group is an oxygen-ether formed with aforementioned straight-chain, branched or cyclic alkyls.

Unless otherwise stated, the term “aryl” as used herein refers to an unsubstituted or substituted monocyclic or polycyclic ring system containing carbon ring atoms. Preferred aryl groups are monocyclic or bicyclic 6-10 membered aromatic ring systems. Phenyl and naphthyl are preferred aryl groups, and the former is the most preferred.

Unless otherwise stated, the term “heteroaryl” as used herein means an unsubstituted or substituted stable 5- or 6-membered monocyclic aromatic ring system, or an unsubstituted or substituted 9- or 10-membered benzo-fused heteroaromatic ring system or bicyclic heteroaromatic ring system. Preferred carbon atoms and 1-4 heteroatoms selected from N, O or S are selected, and the nitrogen or sulfur heteroatoms may be optionally oxidized, and the nitrogen heteroatoms may be optionally selected for quaternization. Heteroaryl can be attached to any heteroatom or carbon atom to form a stable structure. Examples of heteroaryl include, but are not limited to, thienyl, furyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzoisoxazolyl, Benzoxazolyl, benzopyrazolyl, benzothiazolyl, benzothiadiazolyl, Benzotriazolyl adenine, quinolinyl or isoquinolinyl.

The term “cycloalkyl” refers to cyclic saturated alkyl chains with 3-12 carbon atoms, such as cyclopropyl, cyclobutyl, cyclobutyl, cyclobutyl.

The term “substituted” refers to a group in which one or more hydrogen atoms are each independently substituted with the same or different substituents. Typical substituents include but are not limited to halogen (F, Cl, Br or I), C₁₋₈ alkyl, C₃₋₁₂ cycloalkyl, —OR¹, SR¹, ═O, ═S, —C(O)R¹, —C(S)R¹, ═NR¹, —C(O)OR¹, —C(S)OR¹, —NR¹R², —C(O)NR¹R², cyano, nitro, —S(O)₂R¹, —OS(O₂)OR¹, —OS(O)₂R¹, —OP(O)(OR¹)(OR²); wherein R¹ and R² are independently selected from —H, lower alkyl, and lower halogenated alkyl. In some implementations, the substituents are independently selected from —F, —Cl, —Br, —I, —OH, trifluoromethoxy, ethoxy, propoxy, isopropoxy, n-butoxy, Isobutoxy, t-butoxy, —SCH₃, —SC₂H₅, formyl, —C(OCH₃), cyano, nitro, CF₃, —OCF₃, amino, dimethylamino, methylthio, sulfonyl and acetyl.

As used herein, the term “composition” is intended to cover products that contain specific ingredients in particular quantity, as well as any product that is produced directly or indirectly from a combination of specific ingredients in particular quantity. Therefore, a pharmaceutical composition containing the compounds of the present invention as an active ingredient and methods for preparing the compounds of the present invention are also part of the present invention. In addition, some crystalline forms of the compounds may exist as polymorphs, and are therefore intended to be included in the present invention.

In addition, some compounds may form solvates (ie, hydrates) or common organic solvents with water, and these solvates are also included in the scope of the present invention.

Embodiments of substituted alkyl groups include, but are not limited to, 2-aminoethyl, 2-hydroxyethyl, pentachloroethyl, trifluoromethyl, methoxymethyl, pentafluoroethyl, and piperazinylmethyl.

Embodiments of substituted alkoxy include, but are not limited to, aminomethoxy, tetrafluoromethoxy, 2-diethylaminoethoxy, 2-ethoxycarbonylethoxy, 3-hydroxypropoxy.

The compounds of the present invention may also exist in the form of pharmaceutically acceptable salts. For use in medicine, the salts of the compounds in the present invention refer to a non-toxic “pharmaceutically acceptable salts”. The forms of pharmaceutically acceptable salts include pharmaceutically acceptable acidic/anionic or basic/cationic salts. The pharmaceutically acceptable acid/anionic salts usually take the form of protonation of inorganic or organic acid for basic nitrogen. Representative organic or inorganic acids include hydrochloric acid, hydrobromic acid, hydroiodic acid, perchloric acid, sulfuric acid, nitric acid, phosphoric acid, acetic acid, propionic acid, glycolic acid, lactic acid, succinic acid, maleic acid, fumaric acid, apple Acid, tartaric acid, citric acid, benzoic acid, mandelic acid, methanesulfonic acid, oxyethylsulfonic acid, benzenesulfonic acid, oxalic acid, pamoic acid, 2-haphthalenesulfonic acid, p-toluenesulfonic acid, cyclohexane sulfamic acid, Salicylic acid, saccharin or trifluoroacetic acid. Pharmaceutically acceptable alkaline/cationic salts include, but are not limited to, aluminum, calcium, chloroprocaine, choline, diethanolamine, ethylenediamine, lithium, magnesium, potassium, sodium, and zinc.

The prodrugs of the compounds of the present invention are included in the protection scope of the present invention. Generally, the prodrugs refer to functional derivatives that are easily converted in vivo into desired compounds. Therefore, in the therapeutic method of the present invention, the term “administration” shall include the treatment of various conditions described for specific disclosed compounds, or the use of compounds that may not be specifically disclosed, but are converted into specific compounds in viva after administration. Conventional methods for selecting and preparing suitable derivatives of prodrugs have been documented in books Design of Prodrugs (ed. H. Bundgaard, Elsevier, 1985), etc.

Obviously, the definition of any substituent or variable at a particular position in a molecule is independent of any other position in the molecule. It is easy to understand that ordinary technicians in the field can select the substituent or substituted form of the compound of the present invention through the existing technical means and the method described in the present invention to obtain chemically stable and easily synthesized compounds. The mentioned compounds of the present invention may contain one or more asymmetric centers, and may produce diastereoisomers and optical isomers from this. The present invention includes all possible diastereoisomers and their racemic mixtures, their substantially pure resolved enantiomers, all possible geometric isomers and their pharmaceutically acceptable salts.

The above Formula I does not exactly define the three-dimensional structure of a certain position of the compound. The present invention includes all stereoisomers of the compounds shown by Formula I and their pharmaceutically acceptable salts. Further, mixtures of stereoisomers and isolated specific stereoisomers are also included in the present invention. In the synthetic process of preparing such compounds, or in the process of using racemization or epimerization known to ordinary technicians in the field, the product obtained may be a mixture of stereoisomers.

When the compounds shown by Formula I have tautomers, unless otherwise stated, the present invention includes any possible tautomers, their pharmaceutically acceptable salts and mixtures.

When the compounds shown by Formula I and their pharmaceutically acceptable salts have solvate or polymorph, the present invention includes any possible solvate and polymorph. There are no special restrictions on the type of solvents that form the solvate, provided that the solvent is pharmacologically acceptable. Water, ethanol, propanol, acetone and other similar solvents can all be used.

The term “pharmaceutically acceptable salt” refers to a salt prepared from a pharmaceutically acceptable non-toxic base or acid. When the compounds provided by the present invention are acids, the corresponding salts can be conveniently prepared from pharmaceutically acceptable non-toxic bases, including inorganic bases and organic bases. Salts derived from inorganic bases include salts from such as aluminum, ammonium, calcium, copper (high and low valence), ferric, ferrous, lithium, magnesium, manganese (high and low valence), potassium, sodium, zinc. Salts from ammonium, calcium, magnesium, potassium and sodium are preferred. Pharmaceutically acceptable non-toxic organic bases that can be derived into salts include primary amines, secondary mines and tertiary amines, as well as cyclamines and amines containing substituents, such as naturally occurring and synthetic amines containing substituents. Other pharmaceutically acceptable non-toxic organic bases capable of forming salts include ion exchange resins and arginine, betaine, caffeine, choline, N,N′-dibenzylethylenediamine, diethylamine, 2-Diethylaminoethanol, 2-dimethylaminoethanol, ethanolamine, ethylenediamine, N-ethylmorpholine, N-ethylpiperidine, reductive glucosamine, glucosamine, histidine, hydrabamine, isopropylamine, lysine, methylglucamine, morpholine, piperazine, piperidine, polyamine resin, procaine, purine, theobromine, triethylamine, trimethylamine, tripropylamine, tromethamine, etc.

When the compounds provided by the present invention are bases, the corresponding salts can be conveniently prepared from pharmaceutically acceptable non-toxic acids, including inorganic acids and organic acids. Such acids include, for example, acetic acid, benzenesulfonic acid, benzoic acid, camphorsulfonic acid, citric acid, ethanesulfonic acid, formic acid, fumaric acid, gluconic acid, glutamic acid, hydrobromic acid, hydrochloric acid, isethionic acid, Lactic acid, maleic acid, malic acid, mandelic acid, methanesulfonic acid, mucic acid, nitric acid, pamoic acid, pantothenic acid, phosphoric acid, succinic acid, sulfuric acid, tartaric acid and p-toluenesulfonic acid, etc. The better above-mentioned acids are citric acid, hydrobromic acid, formic acid, hydrochloric acid, maleic acid, phosphoric acid, sulfuric acid and tartaric acid. The preferable above-mentioned acids are formic acid, hydrochloric acid.

Since the compounds shown by Formula I will be used as medicines, it is preferable to use a certain purity, e.g., at least 60% purity, a more suitable purity of at least 75%, and a particularly suitable purity of at least 98% (% is a weight ratio).

The pharmaceutical compositions provided by the present invention include the compounds used for active components (or their pharmaceutically acceptable salts) shown by Formula I, a kind of pharmaceutically acceptable excipient, and other optional therapeutic components or ingredients. Although in any given case, the most suitable administrations for active ingredient depend on the particular subject to be administered, the nature of the subject and the severity of the disease, the pharmaceutical compositions of the present invention include pharmaceutical compositions being suitable for the oral, rectal, topical and parenteral administration (including subcutaneous administration, intramuscular injection, and intravenous administration). The pharmaceutical compositions of the present invention can be conveniently prepared by the existence of unit dosage forms known in the field and by any preparation methods known in the pharmaceutical field. In fact, according to conventional drug mixing technology, the compounds of Formula I of the present invention, or prodrugs, or metabolite, or pharmaceutically acceptable salts can be used as an active component to mix with drug carriers to form pharmaceutical compositions. The mentioned pharmaceutical carrier can have a variety of forms, which depend on the desired mode of administration, for example, oral administration or injection (including intravenous injection). Therefore, the pharmaceutical compositions of the present invention may be a separate unit suitable for oral administration, such as a capsule, wafer capsule or tablet containing a predetermined dose of the active ingredients. Further, the pharmaceutical compositions of the present invention may have the forms of powder, granule, solution, aqueous suspension, non-aqueous liquid, oil-in-water emulsion, or water-in-oil emulsion. Furthermore, in addition to the common dosage forms mentioned above, the compounds shown by Formula I or their pharmaceutically acceptable salts can also be administered by a controlled release method and/or a delivery device. The pharmaceutical compositions of the present invention may be prepared by any pharmaceutical methods. Generally, this method includes the steps of associating the active ingredients with the carriers that make up one or more necessary ingredients. In general, the pharmaceutical compositions are prepared through uniform and well mix of the active ingredients and liquid carriers or finely segmented solid carriers or a mixture of the two. In addition, the product can be easily prepared into the desired appearance.

Therefore, the pharmaceutical compositions of the present invention include pharmaceutically acceptable carriers, compounds shown by Formula I or their stereoisomers, tautomers, polymorphs, solvates, pharmaceutically acceptable salts and lei prodrugs. The drug combination of the compounds shown by Formula I or their pharmaceutically acceptable salts, and one or more other compounds with therapeutic activity, is also included in the pharmaceutical compositions of the present invention.

The drug carrier used in the present invention can be, for example, a solid carrier, a liquid carrier or a gas carrier. Solid carriers include lactose, gypsum powder, sucrose, talc, gelatin, agar, pectin, gum arabic, magnesium stearate, and stearic acid. Liquid carriers include syrup, peanut oil, olive oil and water. The gas carriers include carbon dioxide and nitrogen. When preparing oral pharmaceutical preparations, any pharmacologically convenient medium can be used. For example, water, ethylene glycol, oils, alcohols, flavor enhancers, preservatives, coloring agents, etc; liquid preparations that can be used for oral administration such as suspending agents, elixirs and solutions, carriers such as starches, sugars, microcrystalline cellulose, diluents, granulating agents, lubricants, binders, disintegrants, etc; solid preparations that can be used for oral administration such as powders, capsules and tablets. In consideration of ease of administration, tablets and capsules are preferred for oral preparations, where solid pharmaceutical carriers are used. Standard aqueous or non-aqueous preparation techniques can be used for tablet coating optionally.

The tablet containing the compounds or pharmaceutical compositions of the present invention can be prepared by compression or molding. The compounds or pharmaceutical compositions of the present invention can be made into tablets together with one or more auxiliary components or excipients. The active components with a free-flowing form such as powder or granulesis, are mixed with a binder, lubricant, inert diluent, surfactant or dispersant, then are compressed into tablets in a suitable machine. The powdered compounds or pharmaceutical compositions are soaked with an inert liquid diluent, and then are molded to make tablets in a suitable machine. Ideally, each tablet contains approximately 0.05 mg to 5 g of active components, and each wafer capsule or capsule contains approximately 0.05 mg to 5 g of active components. For example, a formulation intended for oral administration to humans contains about 0.5 mg to 5 g of the active ingredients. The active ingredients are compounded with a suitable and convenient metering auxiliary material which accounts for about 5% to 95% of the total pharmaceutical composition. One unit of dosage form generally contains about 1 mg to 2 g of active ingredients, typically with dosage of 25 mg, 50 mg, 100 mg, 200 mg, 300 mg, 400 mg, 500 mg, 600 mg, 800 mg or 1000 mg.

The present invention provides pharmaceutical compositions suitable for injection, including sterile aqueous solutions or dispersion systems. Further, the above pharmaceutical compositions can be prepared into a sterile powder form for immediate preparation of sterile injections or dispersion solutions. In any case, the final injection form must be sterile, and for easy injection, it must be easy to flow. In addition, the mentioned pharmaceutical compositions must be stable during preparation and storage. Therefore, so it is preferable that the mentioned pharmaceutical compositions should he stored under anti-microbial conditions such as anti-bacteria and anti-fungi contamination. The carrier can be a solvent or dispersion medium, for example, water, ethanol, polyol (such as glycerol, propylene glycol, liquid polyethylene glycol), vegetable oil, and appropriate mixtures.

The pharmaceutical compositions provided by the present invention can be the dosage forms suitable for topical administration, for example, aerosol, emulsion, ointment, lotion, dusting powder or other similar dosage forms. Further, the pharmaceutical compositions provided by the present invention can be the dosage forms suitable for use of transdermal drug delivery device. These preparations can be prepared by conventional processing methods with the use of the compounds shown by Formula I of the present invention, or their pharmaceutically acceptable salts. As an example, emulsion or ointment that meets the desired criteria is prepared by adding about 5 wt % to 10 wt % of hydrophilic material and water.

The pharmaceutical compositions provided by the present invention can take solid as a carrier to be suitable for rectal administration. The unit dose of suppository is the most typical dosage form. Suitable excipients include cocoa butter and other materials commonly used in the field. Suppositories can be conveniently prepared by cooling and moulding the mixture of the pharmaceutical compositions and softened or melted excipients.

In addition to the above excipients, the above formulations may also include, as appropriate, one or more additional excipients, such as thinners, buffering agents, flavoring agents, binders, surfactants, thickeners, lubricants and preservatives (including antioxidants), etc. Further, other auxiliary medicines can also include regulating drugs and penetration enhancers that are isotonic with blood. The pharmaceutical compositions containing the compounds shown by Formula 1, or their pharmaceutically acceptable salts, can be prepared in the form of powder or concentrated solution.

In general, the dosage levels of the drug for the treatment of the conditions or discomforts described above range from 0.01 mg/kg body weight to 150 mg/kg body weight per day, or 0.5 mg to 7 g per patient per day. For example, inflammation, cancer, psoriasis, allergy/asthma, diseases and discomforts of the immune system, and diseases and discomforts of the central nervous system (CNS), are effectively treated at the dosage levels of the drug ranging from 0.01 mg/kg body weight to 50 mg/kg body weight per day, or 0.5 mg to 3.5 g per patient per day.

However, it is understood that lower or higher dosages than those mentioned above may be required. The specific dosage level and treatment plan for any particular patient will depend on many factors, including the activity of the specific compound used, age, weight, overall health, sex, diet, time of administration, route of administration, excretion rate, drug combination and the severity of the specific disease being treated.

These and other aspects will become apparent from the following written description for the invention.

The following examples are provided to better illustrate the present invention. Unless expressly stated otherwise, all portions and percentages are by weight, and all temperature units are in degrees Celsius.

The invention will be described in more detail by means of embodiments. The following embodiments are provided for illustrative purposes without the intention of limiting the present invention in any way. Technicians in the field will easily recognize that various non-critical parameters can be changed or modified to produce essentially the same results. According to at least one of the assay methods described herein, the compounds of the embodiments have been found to have the ability to inhibit L858R, Δ19del, T790M, and C797S.

### EXAMPLES

It should be understood that the preceding general descriptions and the detailed descriptions below are exemplary and illustrative only and are not a restriction on any subject requiring protection. Unless expressly stated otherwise, all portions and percentages are by weight, and all temperature units are in degrees Celsius. The compounds described herein can be obtained from commercial sources or synthesized by conventional methods as shown below using commercially available raw materials and reagents.

The following abbreviations have been used in embodiments:

AcOH: acetic acid;

DIEA: N,N-diisopropylethylamine;

DMF: N,N-dimethylformamide;

DMSO: dimethyl sulfoxide;

EA: ethyl acetate;

HEPES: 4-(2-hydroxyethyl)-1-piperazine ethanesulfonic acid;

LCMS: Liquid Chromatography-Mass Spectrometry;

h or hrs: hours;

Pd/C: Palladium on active carbon;

MeOH: methanol;

n-BuOH: n-butanol

DME: 1,2-Dimethoxyethane

1,4-dioxane: 1,4-dioxane

p-TsOH: p-toluenesulfonic acid

K₂CO₃: Potassium carbonate

K₃PO₄: Potassium phosphate

NMP: N-methyl-2-pyrrolidone;

TLC: Thin Layer Chromatography;

Ra/Ni: Raney Nickel

Pd(OAc)₂: Palladium acetate

Pd(PPh₃)₄: Tetrakis(triphenylphosphine) palladium(0)

Pd(dppt)₂Cl₂CH₂Cl₂: 1,1′-Bis(diphenylphosphino)ferroceue-palladium(II)dichloride dichloromethane complex

Xantphos: 4,5-bis-diphenylphosphanyl-9,9-dimethyl-9h-xanthene.

### Example 1 Synthesis of Compound 1

### (6-((5-Chloro-2-((2-methoxy-5-(1-methyl-1hydro-pyrazol-4-yl)-4-(4-(4-methylpiperazine-1-yl) piperidin-1-yl)phenyl)amino)pyrimidin-4-yl)amino)quinoxalin-5-yl)dimethylphosphine oxide

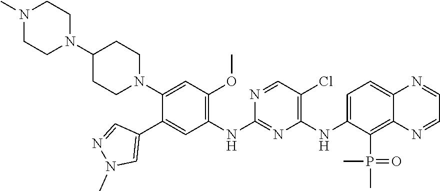 [SMILES: COc1cc(N2CCC(N3CCN(C)CC3)CC2)c(-c2cnn(C)c2)cc1Nc1ncc(Cl)c(Nc2ccc3nccnc3c2P(C)(C)=O)n1]

### Step 1: Synthesis of Compound 1-2

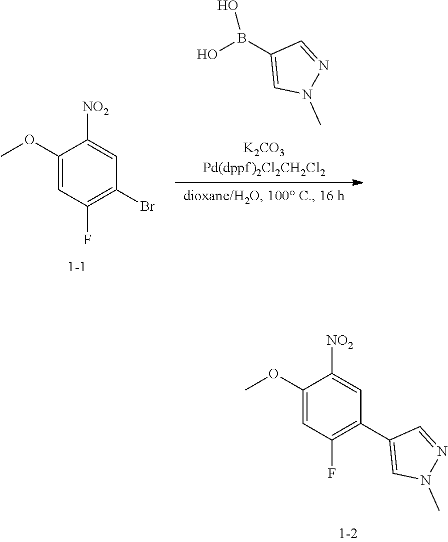

Under nitrogen gas protection, add 1-1 (500 mg) and N-methylpyrazoleboronic acid (450 mg) into a 50 mL single-neck flask, dissolve them in 10 mL dioxane and 2 mL water, and then add Pd(dppf)₂C₁₂CH₂C₁₂ (160 mg) and K₂CO₃ (560 mg) into the flask, then replace nitrogen gas for three times, and keep them for chemical reaction overnight in an oil bath at 100° C. Cool down. Pour the reaction solution into water, extract it with ethyl acetate for three times, and merge, dry and concentrate the organic phase. The obtained crude is slurried with petroleum ether to get 400 mg product 1-2, MS: 252 [M+H]+

### Step 2: Synthesis of Compound 1-3

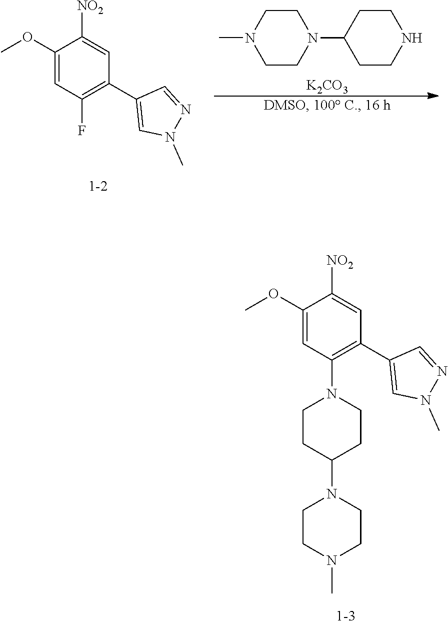

Add 1-2 (400 mg) and 1-methyl-4-(4-piperidinyl)piperazine (146 mg) into a 50 mL single-neck flask, dissolve them in 10 mL DMSO, and then add K₂CO₃ (440 mg) into the flask, and keep them for chemical reaction overnight in an oil bath at 100° C., then process them by the way of cooling down, diluting, washing with water, washing with saturated sodium chloride, drying the organic phase, spinning dry, column chromatography (petroleum ether: ethyl acetate=4:1) to get the product (compound 1-3) 600 mg, MS: 415 [M+H]+

### Step 3: Synthesis of Compound 1-4

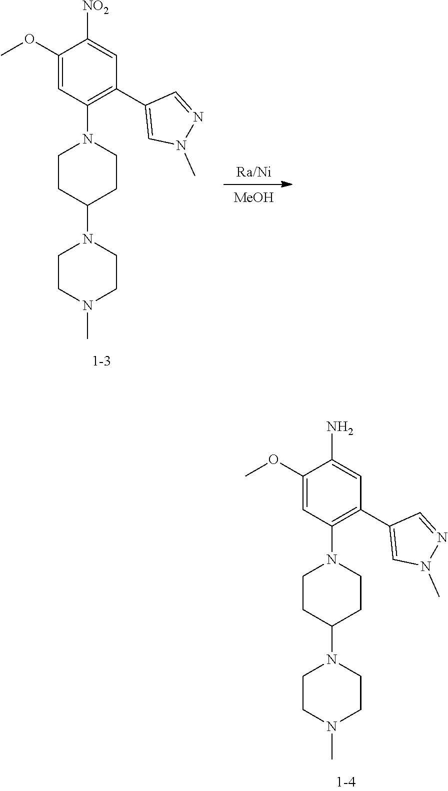 [SMILES: "MeOH" ; COc1cc(N2CCC(N3CCN(C)CC3)CC2)c(-c2cnn(C)c2)cc1[N+](=O)[O-] ; COc1cc(N2CCC(N3CCN(C)CC3)CC2)c(-c2cnn(C)c2)cc1N]

Add 1-3 (200 mg) to a 25 single-neck flask, dissolve them in 5 mL methanol, and then add Raney nickel (20 mg) into the flask, then replace nitrogen gas for three times, and keep them for chemical reaction at 25° C. for 2 h, then filter them and spin them dry to obtain the product (Compound 1-4) 100 mg, proceed to the next reaction directly, MS: 385 [M+H]+

### Step 4: Synthesis of Compound 1-6

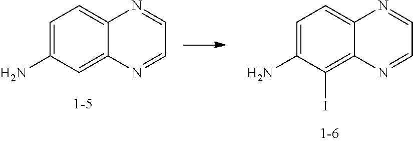 [SMILES: Nc1ccc2nccnc2c1 ; Nc1ccc2nccnc2c1I]

Add 1-5 (2.5 g) to a 200 mL single-neck flask, dissolve them in 75 mL AcOH, add iodine chloride (dissolve iodine chloride in 25 mL AcOH) dropwise into the flask at room temperature, keep them fir chemical react at room temperature for 1 h, concentrate them, then add 100 mL ethyl acetate, and use saturated sodium bicarbonate solution to adjust the pH to 9, then wash it with water, sodium thiosulfate aqueous solution and saturated brine, then process them by the way of drying, filtering, spinning dry to get crude product, then the crude product is slurried with ether to obtain 4 g of a brown-red product, which is directly proceeded to the next reaction, MS: 272 [M+H]+

### Step 5: Synthesis of Compound 1-7

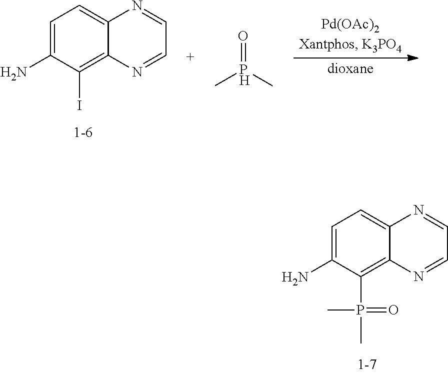

Add compound 1-6 (3.0 g), dimethylphosphine oxide (1.29 g), K₃PO₄ (5.49 g), Pd(OAc)₂ (243.10 mg), Xantphos (1.25 g), and dioxane (20 mL) into a 100 mL reaction flask, raise the temperature to 100° C. under N₂ protection, and then heat and stir for 12 h. Stop the reaction when LCMS monitors the end of the reaction. Add water (50 mL) to the reaction solution to extract it with dichloromethane (3×50 mL), wash the organic phase with saturated brine (3×30 mL), and then dry it with anhydrous sodium sulfate, then separate and purify it with column chromatography (dichloromethane: Methanol=15:1), and then remove the solvent to get the target product 1-6 (2.0 g) as a brown solid, MS: 222 [M+H]⁺

### Step 6: Synthesis of Compound 1-9

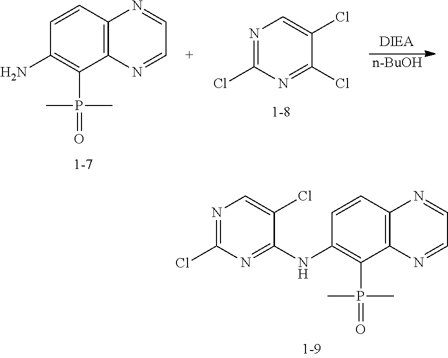

Add compounds 1-7 (1 g), 1-8 (1.64 g), DIEA (1.16 g), and n-BuOH (20 mL) to the reaction flask in sequence, raise the temperature to 120° C., heat and stir for 12 h. Stop the reaction when LCMS monitor the end of the reaction. Add water (50 mL) to the reaction solution to extract it with dichloromethane (3×50 mL), wash the organic phase with saturated brine (3×30 mL), and then dry and concentrate it with anhydrous sodium sulfate, then separate and purify it with Column chromatography (dichloromethane: Methanol=15:1), and then remove the solvent to get the target product 1-9(0.6 g) as a yellow solid. MS:368 [+H]⁺

### Step 7: Synthesis of Compound 1

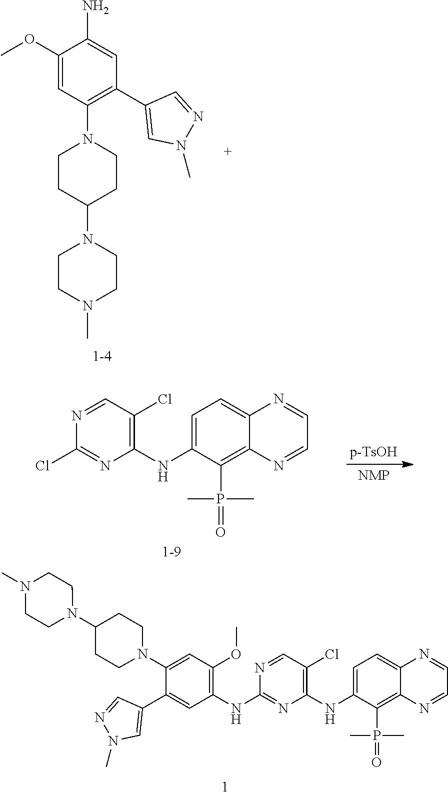 [SMILES: COc1cc(N2CCC(N3CCN(C)CC3)CC2)c(-c2cnn(C)c2)cc1N ; CP(C)(=O)c1c(Nc2nc(Cl)ncc2Cl)ccc2nccnc12 ; COc1cc(N2CCC(N3CCN(C)CC3)CC2)c(-c2cnn(C)c2)cc1Nc1ncc(Cl)c(Nc2ccc3nccnc3c2P(C)(C)=O)n1]

Add 1-4 (100 mg), 1-5 (105 mg) to a 5 mL microwave tube, dissolve them in NMP (1 mL), and then add p-TsOH (95 mg) for chemical react at 140° C. for 1 h, and the system was directly purified with Flash purification system to get the 64.4 mg of product (CH₃CN:H₂O=5%˜50%). MS: 716 [M+H]⁺

### Example 2 Synthesis of Compound 2

### (6-((5-Chloro-2-((4-(4-dimethylamino)piperidine-1-yl)-2-methoxy-5-(1-methyl-1H-pyrazole-4-yl) phenyl)amino)pyrimidine-4-yl)amino)quinoxalin-5-yl)dimethylphosphine oxide

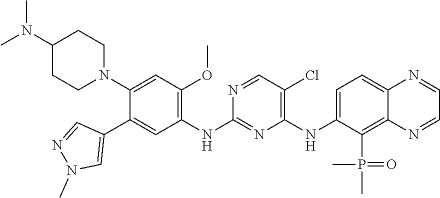 [SMILES: COc1cc(N2CCC(N(C)C)CC2)c(-c2cnn(C)c2)cc1Nc1ncc(Cl)c(Nc2ccc3nccnc3c2P(C)(C)=O)n1]

### Step 1: Synthesis of Compound 2-2

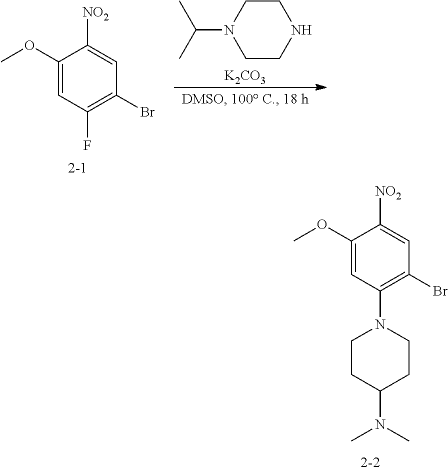

Prepare compound 2-2 as the method described for compound 1-3 using compound 2-1 instead of compound 1-2, N,N-Dimethylpiperidin-4-amine instead of 1-methyl-4-(4-piperidinyl)piperazine. MS: 358 [M+H]⁺

### Step 2: Synthesis of Compound 2-3

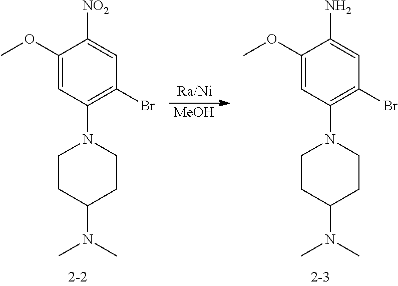 [SMILES: "MeOH" ; COc1cc(N2CCC(N(C)C)CC2)c(Br)cc1[N+](=O)[O-] ; COc1cc(N2CCC(N(C)C)CC2)c(Br)cc1N]

Prepare compound 2-3 as the method described for compound 1-4 using compound 2-2 instead of compound 1-3. MS: 328 [M+H]⁺

### Step 3: Synthesis of Compound 2-4

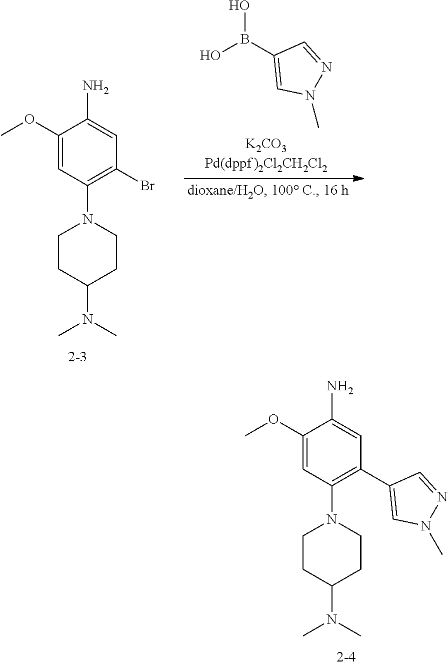

Prepare compound 2-4 as the method described for compound 1-2 using compound 2-3 instead of compound 1-1, MS: 330 [M+H]⁺

### Step 4: Synthesis of Compound 2

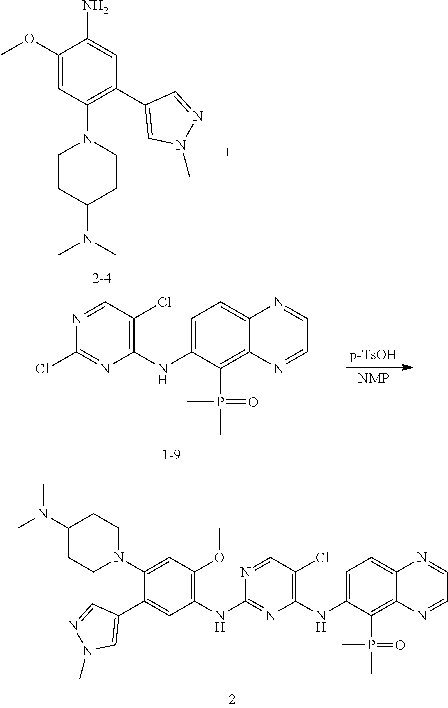 [SMILES: COc1cc(N2CCC(N(C)C)CC2)c(-c2cnn(C)c2)cc1N ; CP(C)(=O)c1c(Nc2nc(Cl)ncc2Cl)ccc2nccnc12 ; COc1cc(N2CCC(N(C)C)CC2)c(-c2cnn(C)c2)cc1Nc1ncc(Cl)c(Nc2ccc3nccnc3c2P(C)(C)=O)n1]

Prepare compound 2 as the method described for compound 1 using compound 2-4 instead of compound 1-4. MS: 661 [M+H]+

### Example 3 Synthesis of Compound 3

### (dimethylamino)piperidine-1-yl)-2-methoxy-5-(1H-pyrazol-4-yl) phenyl)amino)pyrimidine-4-yl)amino)quinoxalin-5-yl)dimethylphosphine oxide

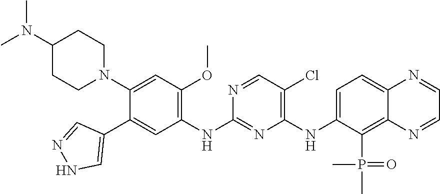 [SMILES: COc1cc(N2CCC(N(C)C)CC2)c(-c2cn[nH]c2)cc1Nc1ncc(Cl)c(Nc2ccc3nccnc3c2P(C)(C)=O)n1]

### Step 1: Synthesis of Compound 3-2

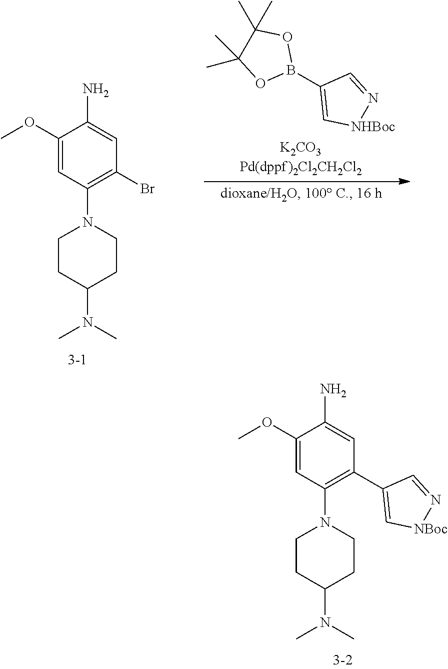

Prepare compound 3-2 as the method described for compound 1-2 using compound 3-1 instead of compound 1-1, compound 1-Boc-4-pyrazole boronic acid instead of compound N-methylpyrazole boronic acid. MS: 416 [M+H]+

### Step 2: Synthesis of Compound 3

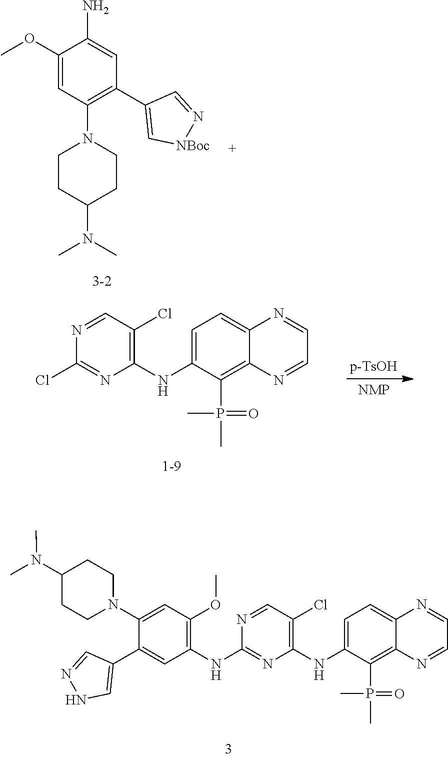 [SMILES: COc1cc(N2CCC(N(C)C)CC2)c(-c2cnn(C(=O)OC(C)(C)C)c2)cc1N ; CP(C)(=O)c1c(Nc2nc(Cl)ncc2Cl)ccc2nccnc12 ; COc1cc(N2CCC(N(C)C)CC2)c(-c2cn[nH]c2)cc1Nc1ncc(Cl)c(Nc2ccc3nccnc3c2P(C)(C)=O)n1]

Prepare compound 3 as the method described for compound 1 using compound 3-2 instead of compound 1-4. MS:647 [M+H]⁺

### Example 4 Synthesis of Compound 4

### (6-((5-Chloro-2-((4-(4-(dimethylamino)piperidine-1-yl)-2-methoxy-5-(1-methyl-1H-pyrazole-4-yl) phenyl)amino)pyrimidine-4-yl)amino)-2,3-dimethylphenyl)dimethylphosphine oxide

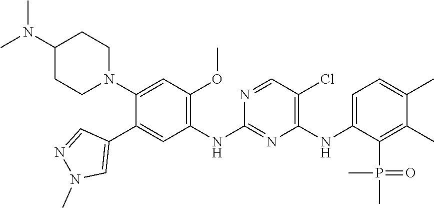

### Step 1: Synthesis of Compound 4-1

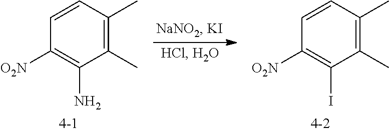

Add compound 4-1 (2.0 g), HCl (10 mL) to the reaction flask, and add NaNO₂ (996.53 mg) aqueous solution (5 mL) dropwise into the reaction flask at 0° C., and continue stirring for 1 h, then add KI (3.00 g) aqueous solution (10 mL), keep it return to room temperature naturally and continue stirring for 1 h. Stop the reaction as TLC monitoring the end of the reaction. Add water (20 mL) to the reaction solution to extract the organic phase with ethyl acetate (3×20 mL), and wash the organic phase with Na₂S₂O₃ (3×20 mL), saturated brine (3×20 mL), and dry it with anhydrous sodium sulfate, then separate and purify it with Column chromatography (n-hexane: ethyl acetate=8:1), and then remove the solvent to get the target compound 4-2 (3.0 g) as a yellow solid.

### Step 2: Synthesis of Compound 4-3

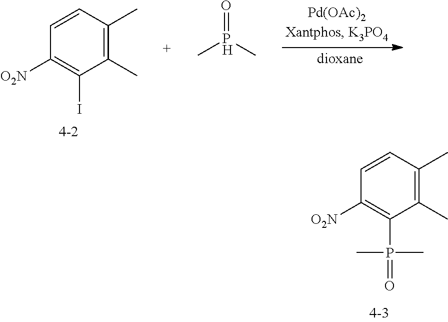

Add compound 4-2 (3.0 g), dimethylphosphine oxide (1.27 g), K₃PO₄ (4.60 g), Pd(OAc) 2 (243.10 mg), Xantphos (1.25 g), dioxane (20 mL) to the reaction flask in sequence, under the protection of N2, raise the temperature to 100° C., and then heat and stir for 12 h. Stop the reaction when LCMS monitoring the end of the reaction. Add water (50 mL) to the reaction solution to extract it with dichloromethane (3×50 mL), then wash the organic phase with saturated brine (3×30 mL), and dry it with anhydrous sodium sulfate, then separate and purify it with Column chromatography (dichloromethane: Methanol=15:1), and then remove the solvent to get the target product 4-3 (2.0 g) as a brown solid. MS: 228 [M+H]⁺

### Step 3: Synthesis of Compound 4-4

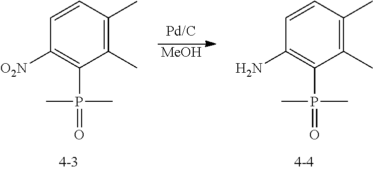

Add compound 4-3 (2.0 g), Pd/C (500 mg), and. MeOH (30 mL) to the reaction flask in sequence, then inject H₂ into it, and stir the reaction solution at room temperature for 3 h. Stop the reaction when LCMS monitoring the end of the reaction. Filter it by suction, rinse with methanol (20 mL), collect the organic phase, then remove the solvent to get the target product 4-4 (1.2 g) as a light brown solid. MS: 198 [M+H]⁺

### Step 4: Synthesis of Compound 4-5

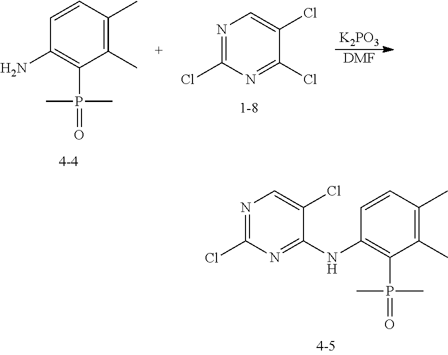

Add compound 4-4 (1 g), 1-8 (1.40 g), K₂CO₃ (1.40 g), and DMF (20 mL) to the reaction flask in sequence, raise the temperature to 100° C., and then heat and stir for 12 h. Stop the reaction when LCMS monitoring the end of the reaction. Pour the reaction solution into water (50 mL) and extract it with dichloromethane (3×50 mL), then tWash the organic phase with saturated brine (3×30 mL), then dry it with anhydrous sodium sulfate, then separate and purify it with Column chromatography (dichloromethane: Methanol=15:1), and then remove the solvent to get the target compound 4-5 (1.5 g) as a yellow solid. MS:344 [M+H]⁺

### Step 5: Synthesis of Compound 4

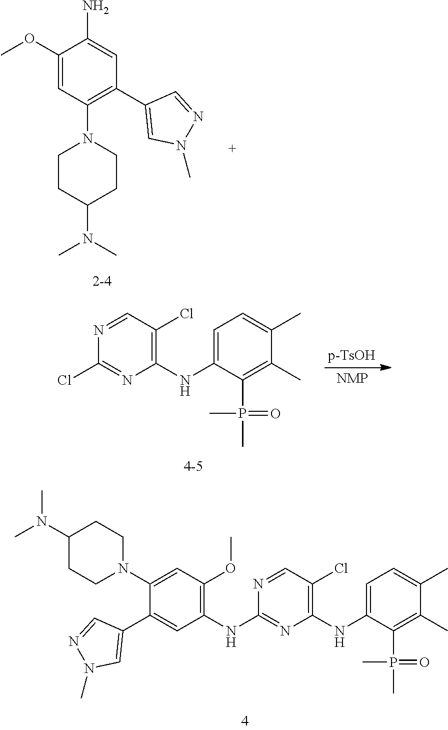

Prepare compound 5 as the method described for compound 1 using compound 2-4 instead of compound 1-4, compound 4-5 instead of compound 1-9. MS: 637 [M+H]⁺

### Example 5 Synthesis of Compound 5 (36560)

### (6-((2-((4-([1,4′-Dipiperidine]-1′-yl)-2-methoxy-5-(1-methyl-1H-pyrazole-4-yl) phenyl)amino)-5-chloropyrimidin-4-yl)amino)quinoxalin-5-yl)dimethylphosphine oxide

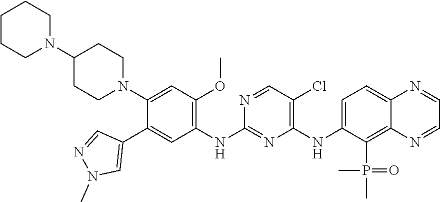 [SMILES: COc1cc(N2CCC(N3CCCCC3)CC2)c(-c2cnn(C)c2)cc1Nc1ncc(Cl)c(Nc2ccc3nccnc3c2P(C)(C)=O)n1]

### Step 1: Synthesis of Compound 5-1

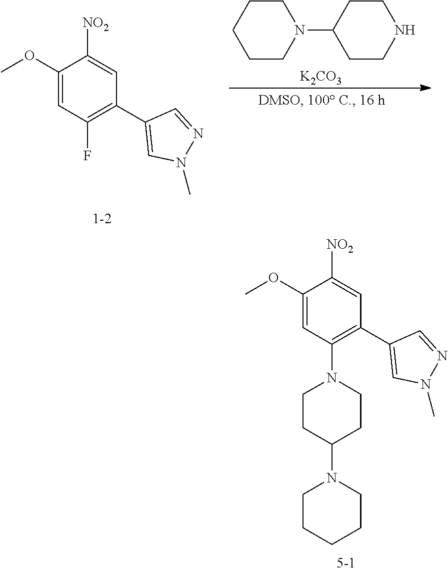

Prepare compound 6-1 as the method described for compound 1-3 using compound 1,4′-bipiperidine instead of compound 1-methyl-4-(4-piperidinyl)piperazine, MS: 400 [M+H]⁺

### Step 2: Synthesis of Compound 5-2

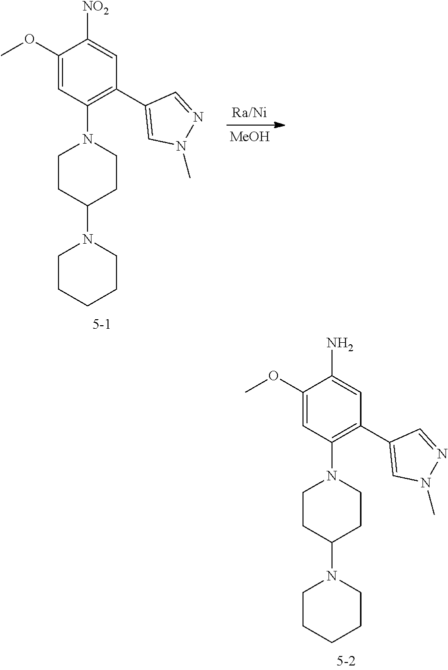 [SMILES: "MeOH" ; COc1cc(N2CCC(N3CCCCC3)CC2)c(-c2cnn(C)c2)cc1[N+](=O)[O-] ; COc1cc(N2CCC(N3CCCCC3)CC2)c(-c2cnn(C)c2)cc1N]

Prepare compound 5-2 as the method described for compound 1-4 using compound 5-1 instead of compound 1-3. MS: 370 [M+H]⁺

### Step 3: Synthesis of Compound 5

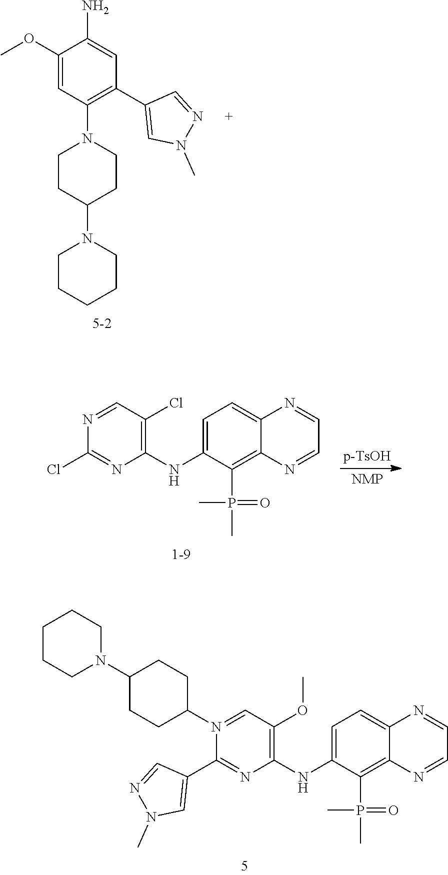

Prepare compound 5 as the method described for compound 1 using compound 5-2 instead of compound 1-4. MS: 701 [M+H]⁺

### Example 6 Synthesis of Compound 6

### (6-((5-Chloro-2-(((2-methoxy-5-(1-methyl-1H-pyrazol-4-yl)-4-(4-(pyrrol-1-yl) piperidine-1-yl) phenyl)amino) pyrimidine-4-yl) amino)-2,3-dimethylphenyl)dimethylphosphine oxide

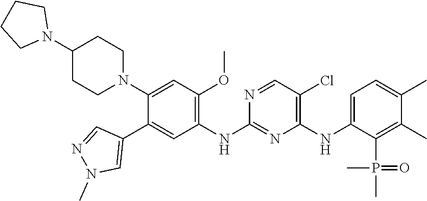 [SMILES: COc1cc(N2CCC(N3CCCC3)CC2)c(-c2cnn(C)c2)cc1Nc1ncc(Cl)c(Nc2ccc(C)c(C)c2P(C)(C)=O)n1]

### Step 1: Synthesis of Compound 6-1

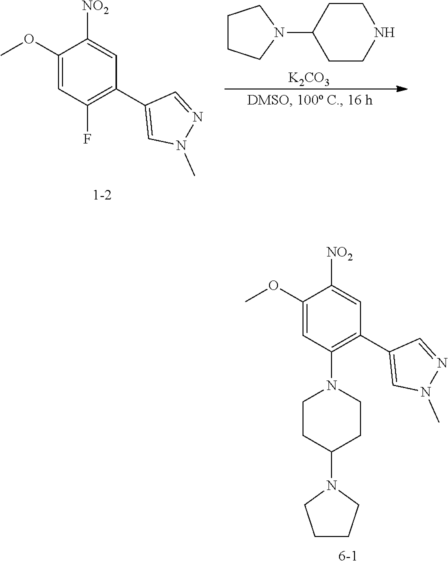

Prepare compound 6-1 as the method described for compound 1-3 using compound 4-pyrrolidine-1-yl-piperidine instead of compound 1-methyl-4-(4-piperidinyl)piperazine. MS: 386 [M+H]⁺

### Step 2: Synthesis of Compound 6-2

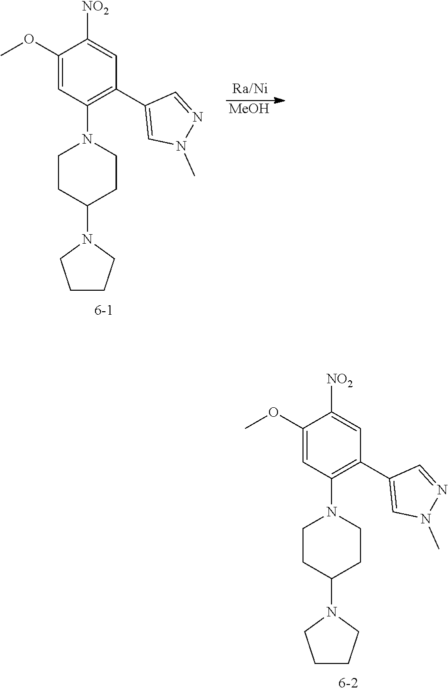 [SMILES: "MeOH" ; COc1cc(N2CCC(N3CCCC3)CC2)c(-c2cnn(C)c2)cc1[N+](=O)[O-] ; COc1cc(N2CCC(N3CCCC3)CC2)c(-c2cnn(C)c2)cc1[N+](=O)[O-]]

Preapre compound 6-2 as the method described for compound 1-4 using compound 6-1 instead of compound 1-3, MS: 356 [M+H]⁺

### Step 3: Synthesis of Compound 6

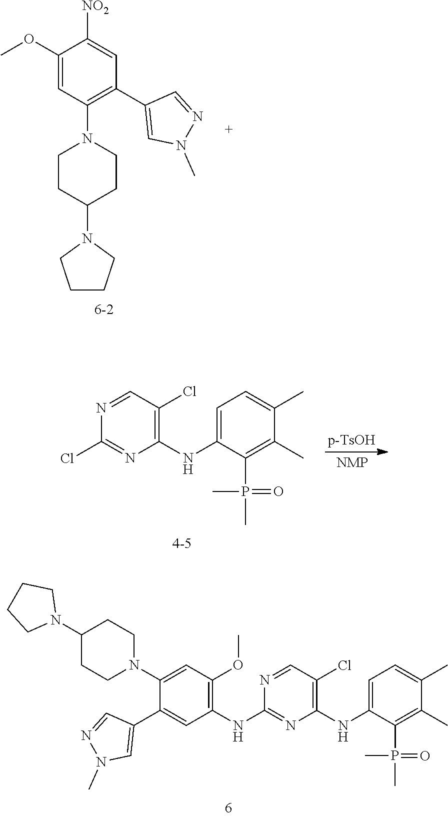 [SMILES: COc1cc(N2CCC(N3CCCC3)CC2)c(-c2cnn(C)c2)cc1[N+](=O)[O-] ; Cc1ccc(Nc2nc(Cl)ncc2Cl)c(P(C)(C)=O)c1C ; COc1cc(N2CCC(N3CCCC3)CC2)c(-c2cnn(C)c2)cc1Nc1ncc(Cl)c(Nc2ccc(C)c(C)c2P(C)(C)=O)n1]

Prepare compound 6 as the method described for compound 4 using compound 6-2 instead of compound 2-4. MS: 663 [M+H]⁺

### Example 7 Synthesis of Compound 7

### (6-((5-chloro-2-((2-methoxy-5-(1-methyl-1H-pyrazol-4-yl)-4-4-(2,7-diaza-spiro[3.5]nonyl-7-yl)phenyl)amino)pyrimidin-4-yl)amino)-2,3-dimethylphenyl)phosphine oxide

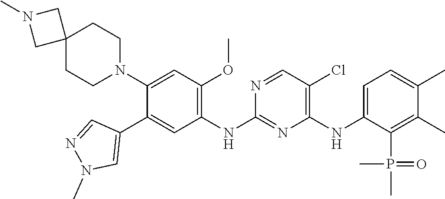

### Step 1: Synthesis of Compound 7-1

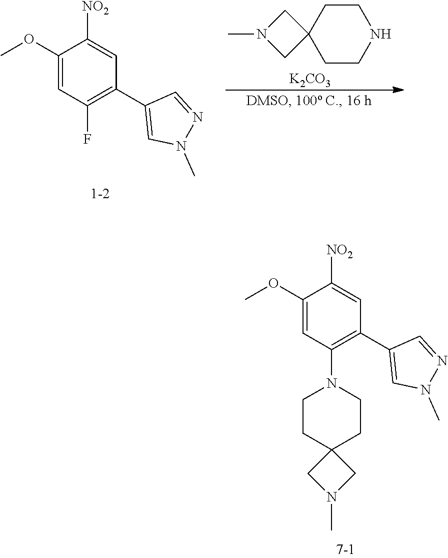

Prepare compound 6-1 as the method described for compound 1-3 using compound 2-methyl-2,7-diaza-spiro[3.5]nonane instead of compound 1-methyl-4-(4-piperidinyl)piperazine, MS: 372 [M+H]⁺

### Step 2: Synthesis of Compound 7-2

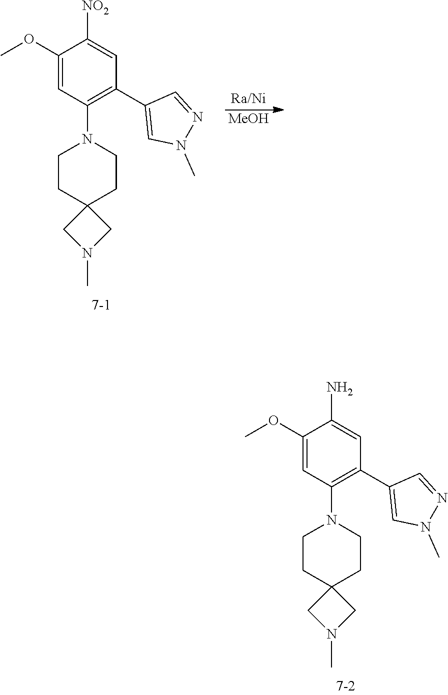 [SMILES: "MeOH" ; COc1cc(N2CCC3(CC2)CN(C)C3)c(-c2cnn(C)c2)cc1[N+](=O)[O-] ; COc1cc(N2CCC3(CC2)CN(C)C3)c(-c2cnn(C)c2)cc1N]

Prepare compound 7-2 as the method described for compound 1-4 using compound 7-1 instead of compound 1-3. MS: 342 [M+H]⁺

### Step 3: Synthesis of Compound 6

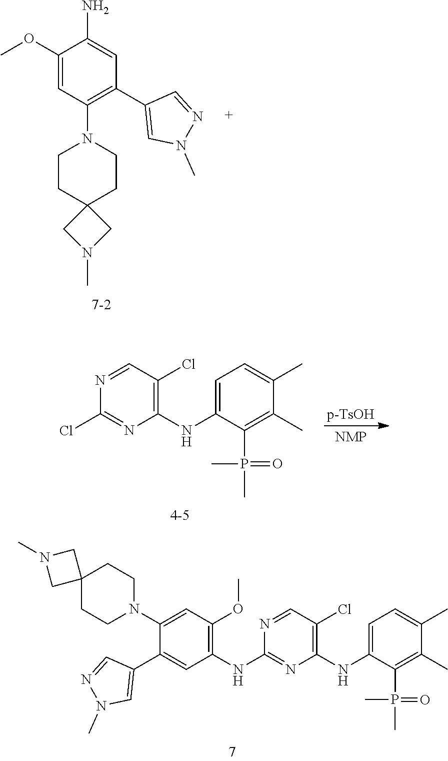

Prepare compound 7 as the method described for compound 4 using compound 7-2 instead of compound 2-4. MS: 649 [M+H]⁺

### Example 8 Synthesis of Compound 8

### (R)-(6-((5-chloro-2-((2-methoxy-5-(1-methyl-1H-pyrazol-4-yl)-4-(octahydro-2hydro-Pyridine[1,2-a]pyrazine-2-yl)phenyl)amino)pyrimidine-4-yl)amino)-2,3-dimethylphenyl)dimethylphosphine oxide

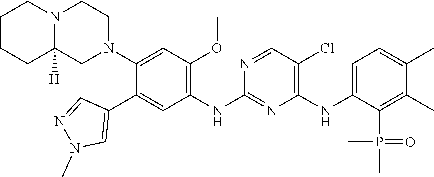 [SMILES: COc1cc(N2CCN3CCCC[C@@H]3C2)c(-c2cnn(C)c2)cc1Nc1ncc(Cl)c(Nc2ccc(C)c(C)c2P(C)(C)=O)n1]

### Step 1: Synthesis of Compound 8-1

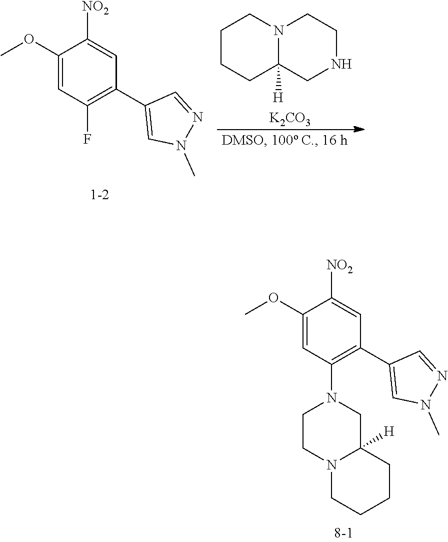

Prepare compound 8-1 as the method described for compound 1-3 using compound (R)-Octahydro-1H-pyrido[1,2-a]pyrazine instead of compound 1-methyl-4-(4-piperidinyl)piperazine. MS: 372[M+H]⁺

### Step 2: Synthesis of Compound 8-2

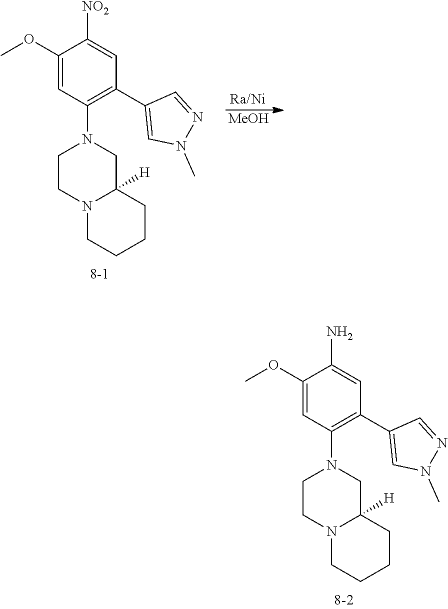 [SMILES: "MeOH" ; COc1cc(N2CCN3CCCC[C@@H]3C2)c(-c2cnn(C)c2)cc1[N+](=O)[O-] ; COc1cc(N2CCN3CCCC[C@@H]3C2)c(-c2cnn(C)c2)cc1N]

Prepare compound 8-2 as the method described for compound 1-4 using compound 8-1 instead of compound 1-3. MS: 342 [M+H]⁺

### Step 3: Synthesis of Compound 8

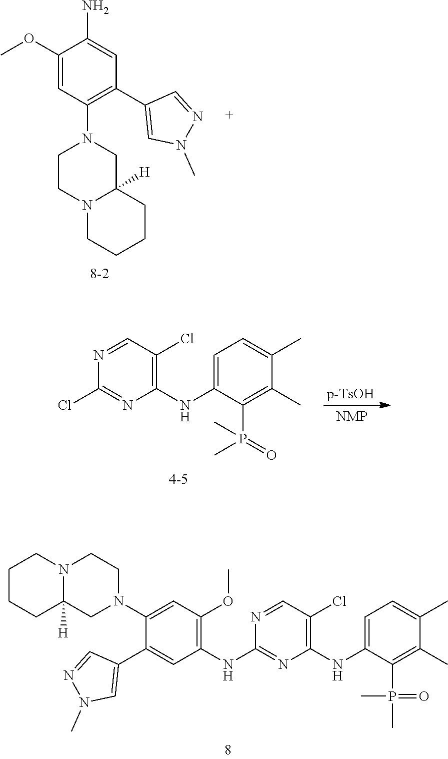 [SMILES: COc1cc(N2CCN3CCCC[C@@H]3C2)c(-c2cnn(C)c2)cc1N ; Cc1ccc(Nc2nc(Cl)ncc2Cl)c(P(C)(C)=O)c1C ; COc1cc(N2CCN3CCCC[C@@H]3C2)c(-c2cnn(C)c2)cc1Nc1ncc(Cl)c(Nc2ccc(C)c(C)c2P(C)(C)=O)n1]

Prepare compound 8 as the method described for compound 4 using compound 8-2 instead of compound 2-4. MS: 649 [M+H]⁺

### Example 9 Synthesis of Compound 9

### (6-((5-chloro-2-((2-methoxy-5-(1-methyl-1hydra-pyrazole)-4-morpholinephenyl) amino)pyrimidine-4-amino)quinoxaline-5-dimethylphosphine oxide

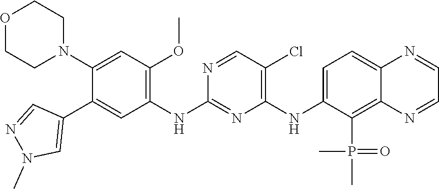 [SMILES: COc1cc(N2CCOCC2)c(-c2cnn(C)c2)cc1Nc1ncc(Cl)c(Nc2ccc3nccnc3c2P(C)(C)=O)n1]

### Step 1: Synthesis of Compound 9-2

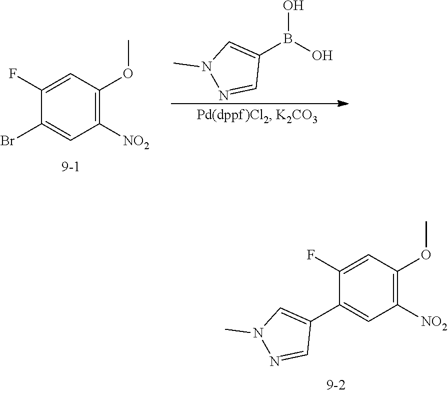

Add 9-1 (0.4 g) and N-methylpyrazole boronic acid (399 mg) to a flask with stirring nitrogen protection, dissolve them in 10 mL dioxane and 2 mL water, and then add Pd(dppf) to it Cl₂ (130 mg) and K₂CO₃ (265 mg) into the flask, then replace nitrogen gas for three times and transfer them into an oil bath at 100° C. to have chemical reaction overnight. when the raw materials for chemical reaction are exhausted and the main peak was the product under LCMS monitoring, stop the chemical reaction and lower the temperature of reaction solution to room temperature. Pour the reaction solution into 50 mL of water, then extractit with 30 mL of ethyl acetate for three times, and the organic phases were merged, dried, and concentrated. The crude product was purified by the mixing with a silica gel column (petroleum ether: ethyl acetate from 0 to 80%) to obtain 400 mg of product. MS: 252 [M+H]⁺

### Step 2: Synthesis of Compound 9-3

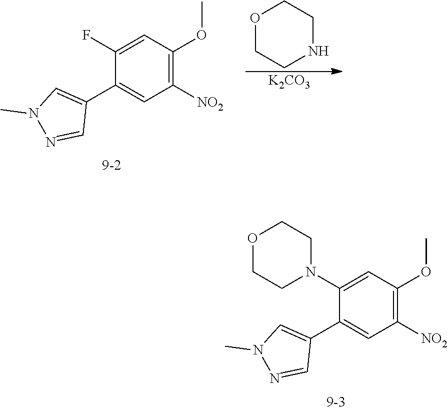

Add 9-2 (0.4 g) and morpholine (278 mg) to a flask with stirring nitrogen protection, dissolve them in 10 mL DMSO, then add K₂CO₃ (441 mg) to the flask, then replace nitrogen gas for three times and transfer them into an oil bath at 90° C. to have chemical reaction overnight. When LCMS monitored that the raw materials for chemical reaction were exhausted and the main peak was the product, stop the chemical reaction and lower the temperature of reaction solution to room temperature. Pour the reaction solution into 60 mL of water, then extractit with 40 mL of ethyl acetate for three times, and the organic phases were merged, dried, and concentrated.

The crude product was purified by the mixing with a silica gel column (petroleum ether: ethyl acetate from 0 to 100%) to obtain 400 mg of product. MS: 319 [M+H]⁺

### Step 3: Synthesis of Compound 9-4

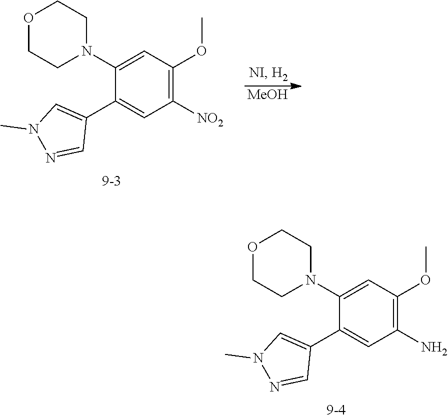

Add compound 9-3 (400 mg) into 15 mL of tetrahydrofuran and 5 ml of methanol in a round bottom flask with a stirring device, and then add 0.25 g of Raney Ni into flask, then replace nitrogen gas for three times, and keept them for chemical reaction at room temperature for 3 hours. When LCMS monitored that the raw materials for chemical reaction were exhausted and the main peak was the product, stop the chemical reaction and filter the reaction solution by adding diatomaceous earth, and then the filtrate was concentrated and dried by rotary evaporation. The crude product was not subjected to further purification treatment, and was directly used in the next reaction to obtain 300 mg of crude compound 9-4. MS: 289 [M+H]⁺

### Step 4: Synthesis of Compound 9

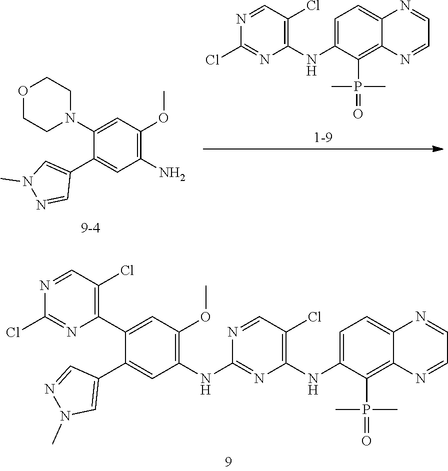

Add compounds 9-4 (78 mg) and 1-9 (100 mg) into 10 mL of n-butanol in the sealed tube with magneton, and then add 28 mg of p-toluenesulfonic acid, and keep them for chemical reaction in an oil bath for 16 hours at 110° C., When LCMS monitored that the raw materials for chemical reaction were exhausted and the main peak was the product, stop the chemical reaction and lower the temperature of reaction solution to room temperature. Concentrate the reaction solution, and then separate it with 0-30% H₂O: MeOH in reverse phase to obtain 60 mg of product. MS: 620 [M+H]⁺

Compound 9: ¹H NMR (500 MHz, DMSO-d₆) δ 11.49 (s, 1H), 11.32 (s, 1H), 8.93 (s, 1H), 8.87 (d, 2H), 8.31 (s, 1H), 8.10 (s, 1H), 7.80 (s, 1H), 7.59 (s, 1H), 6.87 (s, 1H), 3.83 (s, 6H), 3.77 (m, 4H), 2.88 (s, 3H), 2.02-2.88 (m, 8H).

### Example 10 Synthesis of Compound 10

### (6-((5-Bromo-2-methoxy-5-(1-methyl-1H-pyrazol-4-yl)-4-(4-(4-methylpiperazine-1-yl) piperidine-1-yl)phenyl)amino)pyrimidine-4-yl)amino)quinoxalin-5-yl)dimethylphosphine oxide

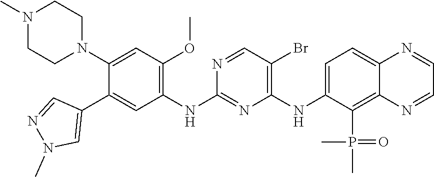 [SMILES: COc1cc(N2CCN(C)CC2)c(-c2cnn(C)c2)cc1Nc1ncc(Br)c(Nc2ccc3nccnc3c2P(C)(C)=O)n1]

### Step 1: Synthesis of Compound 10-2

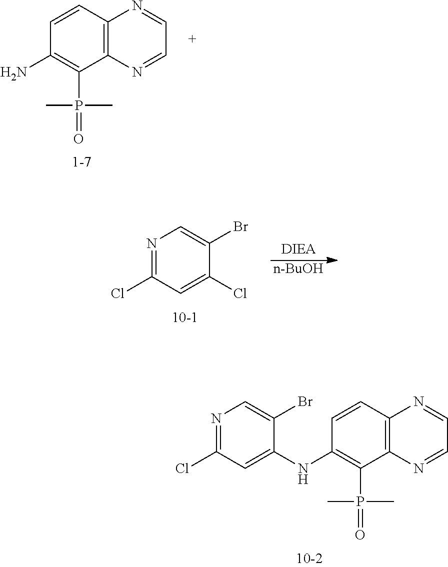

Prepare compound 10-2 as the method described for compound 1-9 using compound 10-1 instead of compound 1-8. MS: 412 [M+H]⁺

### Step 2: Synthesis of Compound 10

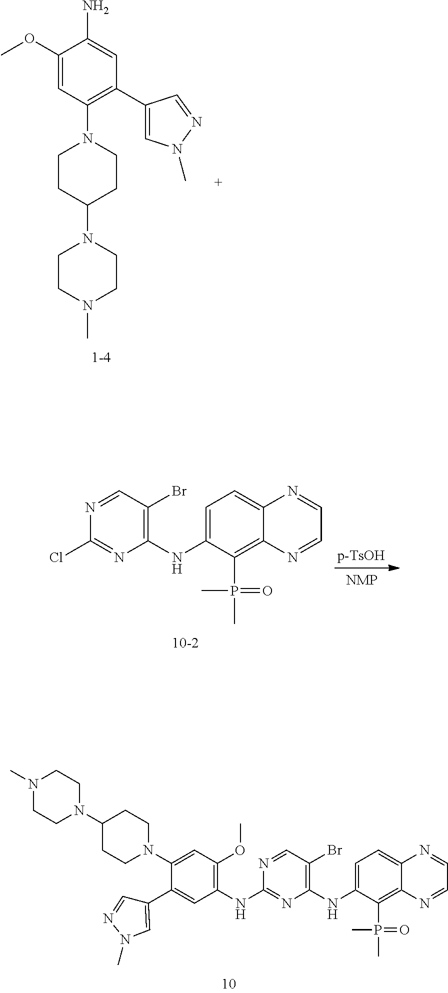 [SMILES: COc1cc(N2CCC(N3CCN(C)CC3)CC2)c(-c2cnn(C)c2)cc1N ; CP(C)(=O)c1c(Nc2nc(Cl)ncc2Br)ccc2nccnc12 ; COc1cc(N2CCC(N3CCN(C)CC3)CC2)c(-c2cnn(C)c2)cc1Nc1ncc(Br)c(Nc2ccc3nccnc3c2P(C)(C)=O)n1]

Prepare compound 10 as the 1 method described for compound 1 using compound 10-2 instead of compound 1-9. MS: 760 [M+H]⁺

### Example 11 Synthesis of Compound 11

### (6-((2-((4-([1,4′-Bipiperidine]-1′-yl)-2-methoxy-5-(1-methyl-1H-pyrazole-4-yl) phenyl)amino)-5-bromopyrimidin-4-yl)amino)quinoxalin-5-yl)dimethylphosphine oxide

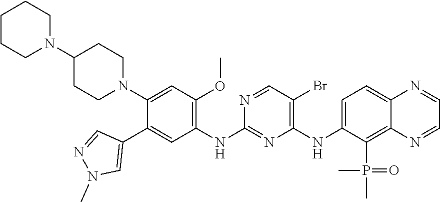 [SMILES: COc1cc(N2CCC(N3CCCCC3)CC2)c(-c2cnn(C)c2)cc1Nc1ncc(Br)c(Nc2ccc3nccnc3c2P(C)(C)=O)n1]

### Step 1: Synthesis of Compound 11

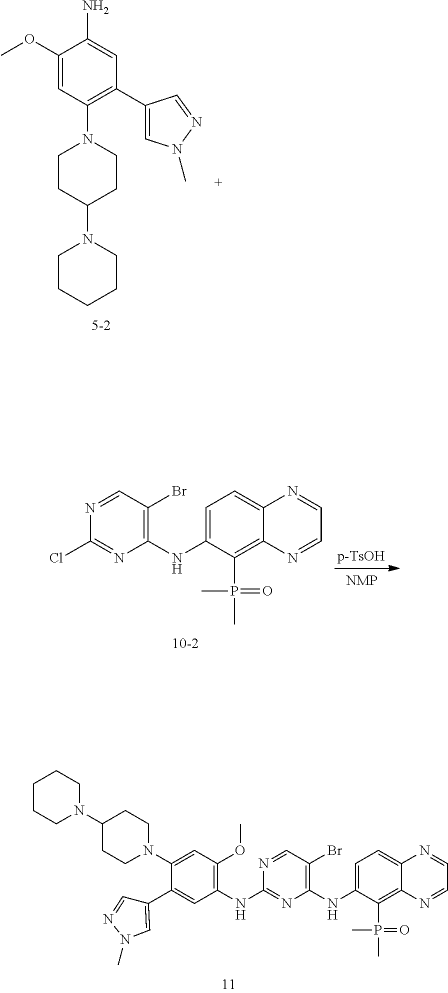 [SMILES: COc1cc(N2CCC(N3CCCCC3)CC2)c(-c2cnn(C)c2)cc1N ; CP(C)(=O)c1c(Nc2nc(Cl)ncc2Br)ccc2nccnc12 ; COc1cc(N2CCC(N3CCCCC3)CC2)c(-c2cnn(C)c2)cc1Nc1ncc(Br)c(Nc2ccc3nccnc3c2P(C)(C)=O)n1]

Prepare compound 11 as the method described for compound 10 using compound 52-instead of compound 1-4. MS: 745 [M+H]⁺

### Example 12 Synthesis of Compound 12

### (6-((5-Bromo-2-methoxy-5-(1-methyl-1H-pyrozol-4-yl)-4-(4-(4-methylpiperazine-1-yl) piperidine-1-yl)phenyl)amino)pyrimidine-4-yl)amino)-3-cyclopropyl-2-methyl)dimethylphosphine oxide

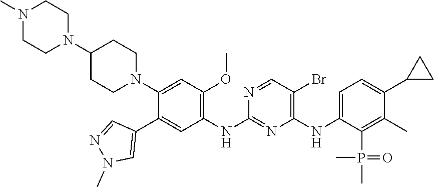 [SMILES: COc1cc(N2CCC(N3CCN(C)CC3)CC2)c(-c2cnn(C)c2)cc1Nc1ncc(Br)c(Nc2ccc(C3CC3)c(C)c2P(C)(C)=O)n1]

### Step 1: Synthesis of Compound 12-2

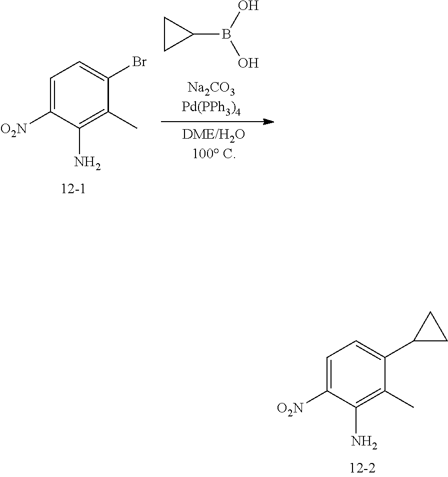

Under nitrogen protection, add 12-1 (5 g) and cyclopropylboronic acid (2.8 g) into a 500 mL single-neck bottle, and then dissolve them in 100 mL ethylene glycol dimethyl ether and 20 mL water, and then add Pd(PPh₃)₄ (2.5 g) and Na₂CO₃ (4.6 g) into the bottle, then replace nitrogen gas for three times, and keep them for chemical reaction in an oil bath at 100° C. for 2 hours. Cool down. Pour the reaction solution into water to extract it with ethyl acetate for three times, then process the organic phases by the way of merging, drying, concentrating and column chromatography (petroleum ether: ethyl acetate=3:1) to get 3.5 g of product. MS: 193 [M+H]⁺

### Step 2: Synthesis of Compound 12-3

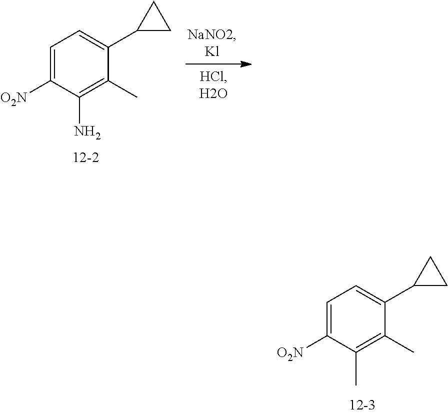

Add compound 12-2 (2.0 g), HCl (10 mL) to a 100 mL three-neck flask, slowly add NaNO₂ (0.86 g) aqueous solution (5 mL) to the reaction flask under 0° C., and continue to stir for 1 h. Add KI (2.59 g) aqueous solution (10 mL), make the temperature rise up naturally and continue stirring for 1 h. Stop the reaction when TLC monitored the end of the reaction. Add water (20 mL) to the reaction solution to extract it with ethyl acetate (3×20 mL), and wash the organic phase with Na₂S₂O₃ (3×20 mL), saturated. brine (3×20 mL) in sequence, and then dry it with anhydrous sodium sulfate, then separate and purify it with Column chromatography (n-hexane: ethyl acetate=8:1), and then remove the solvent to get the target compound 12-3 (2.5 g) as a yellow solid.

### Step 3: Synthesis of Compound 12-4

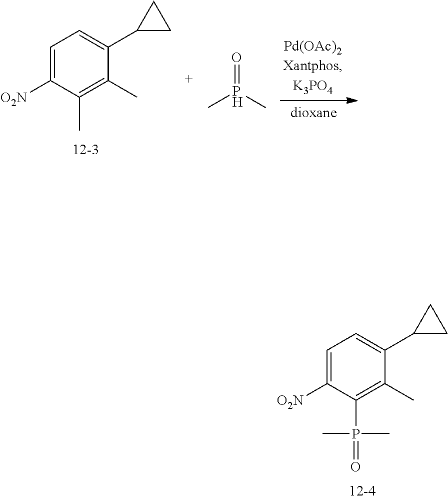

Prepare compound 12-4 as the method described for compound 1-7 using compound 12-4 instead of compound 1-6. MS: 254 [M+H]⁺

### Step 4: Synthesis of Compound 12-5

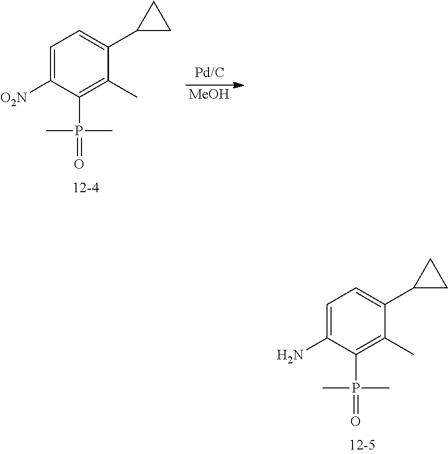 [SMILES: "MeOH" ; Cc1c(C2CC2)ccc([N+](=O)[O-])c1P(C)(C)=O ; Cc1c(C2CC2)ccc(N)c1P(C)(C)=O]

Add 12-4 (1 g) to a 100 mL single-neck flask, and then dissolve it in 50 mL methanol, add Pd/C (200 mg) into the flask, replace hydrogen gas for three times, keep them for chemical reaction at 25° C. for 2 h, then filter and spin them dry to obtain 0.8 g of product which was directly used for the next reaction. MS: 224 [M+H]⁺

### Step 5: Synthesis of Compound 12-6

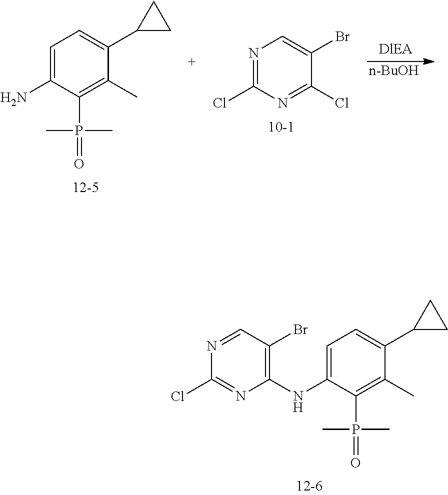

Prepare compound 12-6 as the method described for compound 10-2 using compound 12-5 instead of compound 1-7. MS: 414 [M+H]⁺

### Step 6: Synthesis of Compound 12

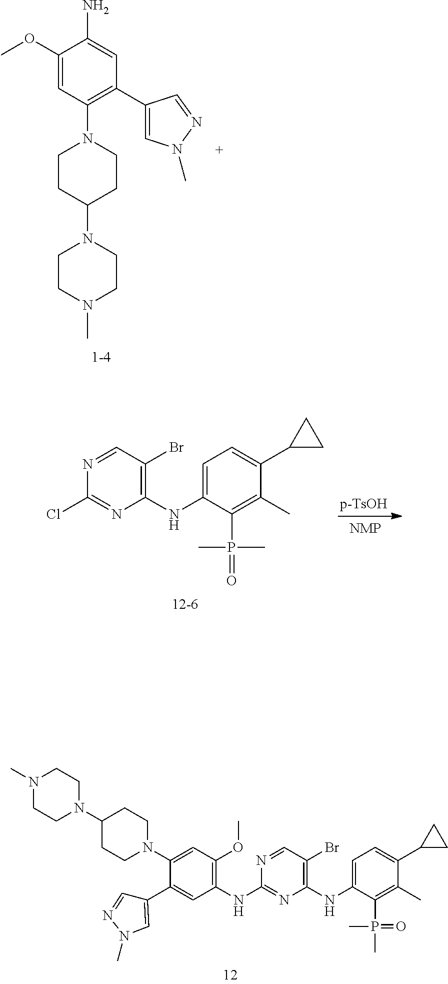 [SMILES: COc1cc(N2CCC(N3CCN(C)CC3)CC2)c(-c2cnn(C)c2)cc1N ; Cc1c(C2CC2)ccc(Nc2nc(Cl)ncc2Br)c1P(C)(C)=O ; COc1cc(N2CCC(N3CCN(C)CC3)CC2)c(-c2cnn(C)c2)cc1Nc1ncc(Br)c(Nc2ccc(C3CC3)c(C)c2P(C)(C)=O)n1]

Prepare compound 12 as the method described for compound 1 using compound 12 instead of compound 1-9. MS: 762 [M+H]+

### Example 13 Synthesis of Compound 13

### (6-((2-((4-([1,4′-Bromopyrimidine]-1′-yl)-2-methoxy-5-(1-methyl-1H-pyrozole-4-yl) phenyl)amino)-5-bromopyrimidin-4-yl)amino)-3-cyclopropyl-2-methylphenyl) dimethylphosphine oxide

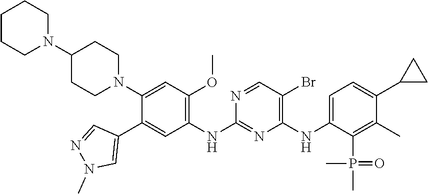 [SMILES: COc1cc(N2CCC(N3CCCCC3)CC2)c(-c2cnn(C)c2)cc1Nc1ncc(Br)c(Nc2ccc(C3CC3)c(C)c2P(C)(C)=O)n1]

### Step 1: Synthesis of Compound 13

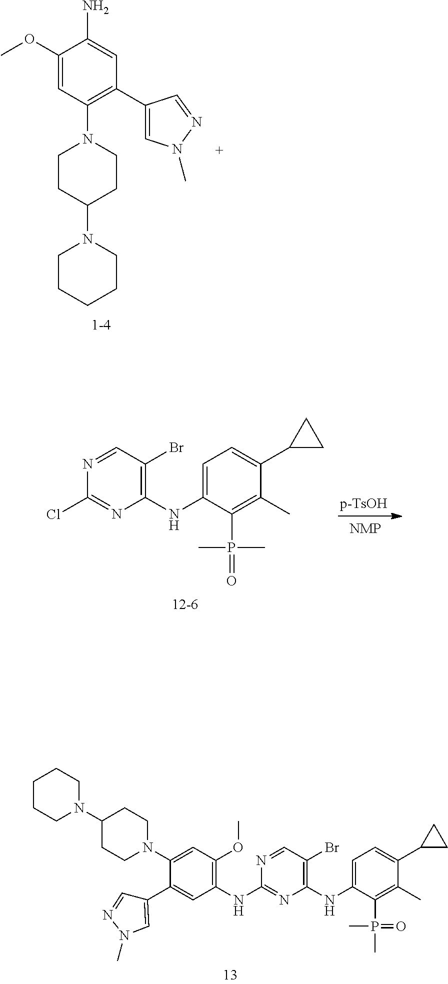 [SMILES: COc1cc(N2CCC(N3CCCCC3)CC2)c(-c2cnn(C)c2)cc1N ; Cc1c(C2CC2)ccc(Nc2nc(Cl)ncc2Br)c1P(C)(C)=O ; COc1cc(N2CCC(N3CCCCC3)CC2)c(-c2cnn(C)c2)cc1Nc1ncc(Br)c(Nc2ccc(C3CC3)c(C)c2P(C)(C)=O)n1]

Prepare compound 13 as the method described for compound 10 using compound 12-6 instead of compound 1-4. MS: 747 [M+H]⁺

### Example 14 Synthesis of Compound 14

### (6-((5-Bromo-2-((2-methoxy-5-(1-methyl-1h-pyrazol-4-yl)-4-(4-(4-methylpiperazine-1-yl) piperidine-1-yl)phenyl)amino)pyrimidine-4-yl)amino)-2,3-dimethylphenyl)dimethylphosphine oxide

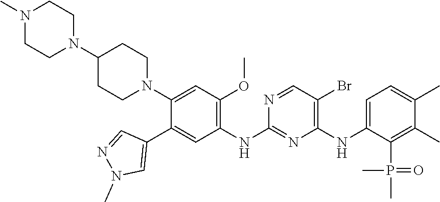

### Step 1: Synthesis of Compound 14-1

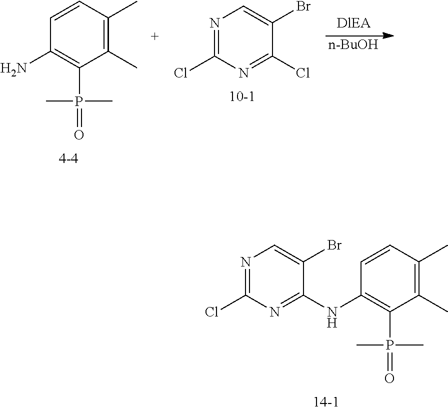

Prepare compound 14-1 as the method described for compound 10-2 using compound 4-4 instead of compound 1-7. MS: 388 [M+H]⁺

### Step 2: Synthesis of Compound 14

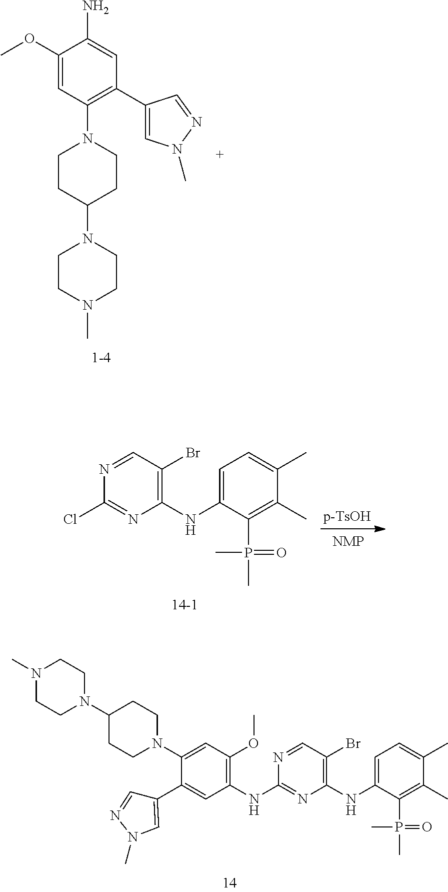

Prepare compound 14 as the method described for compound 1 using compound 14-1 instead of compound 1-9. MS: 736 [M+H]⁺

### Example 15 Synthesis of Compound 15

### (6-((2-((4-([1,4′-Bipiperidine]-1′-yl)-2-methoxy-5-(1-methyl-1H-pyrazole-4-yl) phenyl)amino)-5-bromopyrimidine-4-yl)amino)-2,3-dimethylphenyl)dimethylphosphine oxide

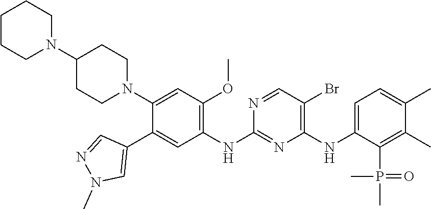 [SMILES: COc1cc(N2CCC(N3CCCCC3)CC2)c(-c2cnn(C)c2)cc1Nc1ncc(Br)c(Nc2ccc(C)c(C)c2P(C)(C)=O)n1]

### Step 1: Synthesis of Compound 15

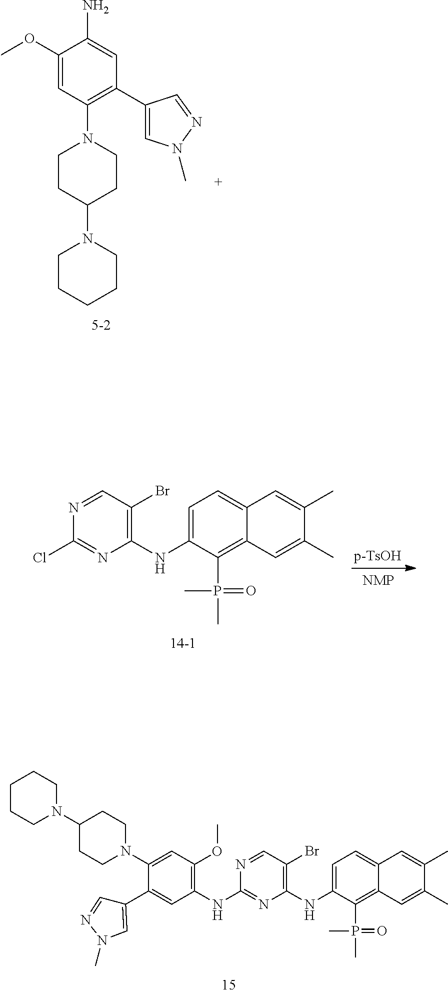 [SMILES: COc1cc(N2CCC(N3CCCCC3)CC2)c(-c2cnn(C)c2)cc1N ; Cc1cc2ccc(Nc3nc(Cl)ncc3Br)c(P(C)(C)=O)c2cc1C ; COc1cc(N2CCC(N3CCCCC3)CC2)c(-c2cnn(C)c2)cc1Nc1ncc(Br)c(Nc2ccc3cc(C)c(C)cc3c2P(C)(C)=O)n1]

Prepare compound 15 as the method described for compound 14 using compound 5-2 instead of compound 1-4. MS: 721 [M+H]⁺

### Example 16 Synthesis of Compound 16

### (6-((5-Bromo-2-((2-methoxy-5-(1-methyl-1hydro-pyrazole)-4-morpholinephenyl) amino)pyrimidine-4-amino)quinoxaline-5-dimethylphosphine oxide

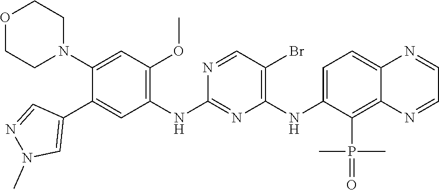 [SMILES: COc1cc(N2CCOCC2)c(-c2cnn(C)c2)cc1Nc1ncc(Br)c(Nc2ccc3nccnc3c2P(C)(C)=O)n1]

### Synthesis of Compound 16

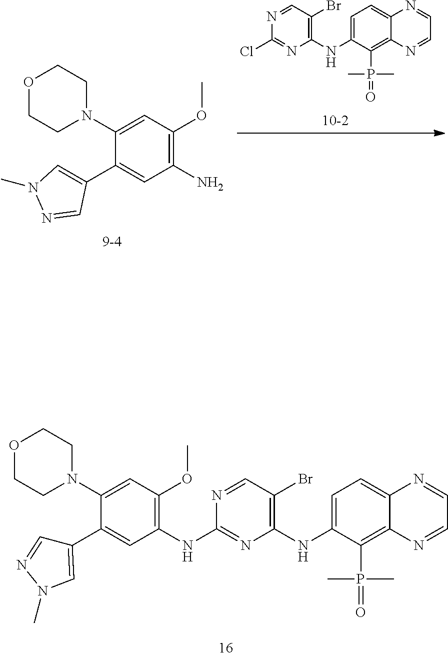

Prepare compound 16 as the method described for compound 9 using compound 10-2 instead of compound 1-9. MS: 664 [M+H]⁺

### Example 17 Synthesis of Compound 17

### (6-((5-Bromo-2-((5-(1-(difluoroethyl)-1hydro-pyrozole-4)-2-methoxy-4-(4-(4-methyl Piperidine-1)piperidine-1-piperidine-1-)phenyl)amino)pyrimidine-4-)amino)quinoxaline-5-dimethylphosphine oxide

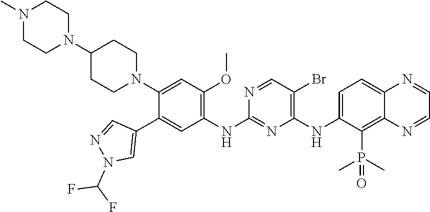 [SMILES: COc1cc(N2CCC(N3CCN(C)CC3)CC2)c(-c2cnn(C(F)F)c2)cc1Nc1ncc(Br)c(Nc2ccc3nccnc3c2P(C)(C)=O)n1]

### Step 1: Synthesis of Compound 17-1

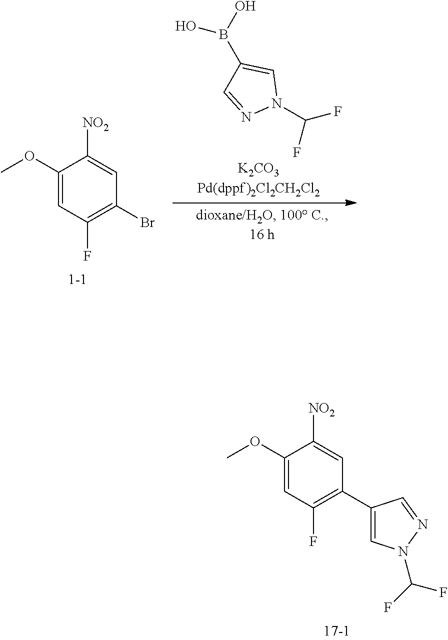 [SMILES: COc1cc(F)c(Br)cc1[N+](=O)[O-] ; OB(O)c1cnn(C(F)F)c1 ; COc1cc(F)c(-c2cnn(C(F)F)c2)cc1[N+](=O)[O-]]

Prepare compound 17-1 as the method described for compound 1-2 using compound N-difluoromethyl pyrazole boronic acid instead of compound N-methylpyrazole boronic acid. MS: 288 [M+H]⁺

### Step 2: Synthesis of Compound 17-2

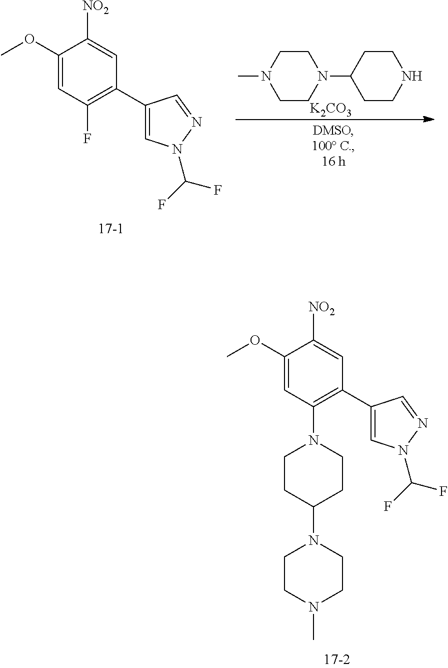

Prepare compound 17-2 as the method described for compound 1-3 using compound 17-1 instead of compound 1-2, MS: 451 [M+H]⁺

### Step 3: Synthesis of Compound 17-3

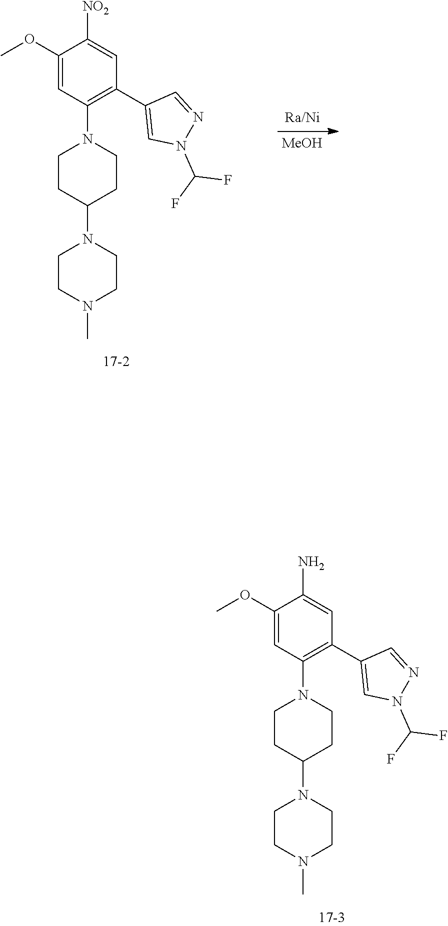 [SMILES: "MeOH" ; COc1cc(N2CCC(N3CCN(C)CC3)CC2)c(-c2cnn(C(F)F)c2)cc1[N+](=O)[O-] ; COc1cc(N2CCC(N3CCN(C)CC3)CC2)c(-c2cnn(C(F)F)c2)cc1N]

Prepare compound 17-3 as the method described for compound 1-4 using compound 17-2 instead of compound 1-3, MS: 421 [M+H]⁺

### Step 4: Synthesis of Compound 17

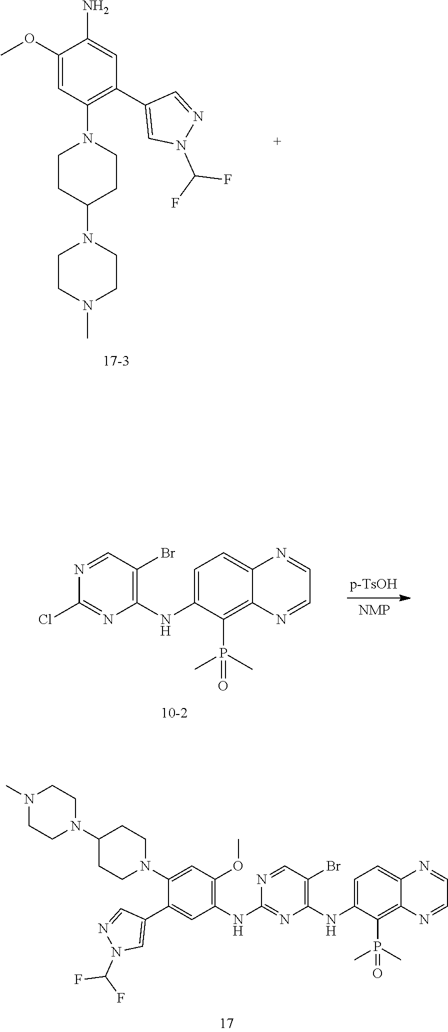

Prepare compound 17 as the method described for compound 1 using compound 17-3 instead of compound 1-4, compound 10-2 instead of compound 1-9, MS: 796 [M+H]⁺

### Example 18 Synthesis of Compound 18

### (R)-(6-((5-Chloro-2-((2-methoxy-5-([1-methyl-1hydro-pyrazol-4-yl]-4-(octahydro-2hydro-pyrozol [1,2-a]pyrazine-2-yl) phenyl)amino)pyrimidin-4-yl)amino)quinoxalin-5-yl)dimethylphosphine oxide

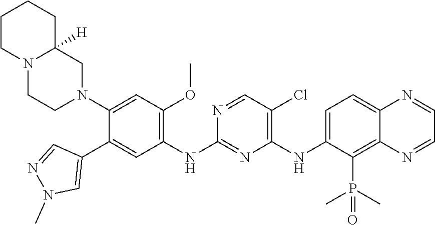 [SMILES: COc1cc(N2CCN3CCCC[C@H]3C2)c(-c2cnn(C)c2)cc1Nc1ncc(Cl)c(Nc2ccc3nccnc3c2P(C)(C)=O)n1]

### Synthesis of Compound 18

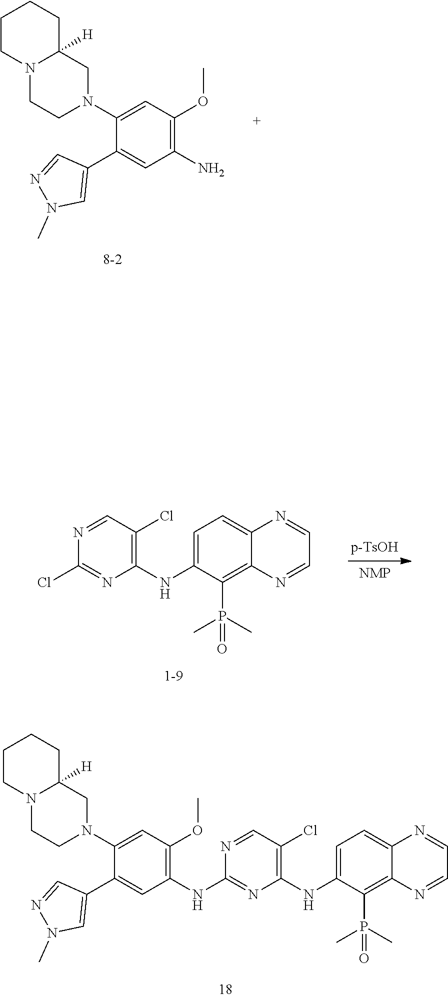

Prepare compound 18 as the method described for compound 1 using compound 8-2 instead of compound 1-4, MS: 673 [M+H]⁺

### Example 19 Synthesis of Compound 19

### (6-((5-Bromo-2-((5-(1-ethyl-1H-pyrazol-4-yl)-2-methoxy)-4-(4-(4-methylpiperazine-1-yl) piperidin-1-yl)phenyl)amino)pyrimidin-4-yl)amino)quinoxalin-5-yl)dimethylphosphine oxide

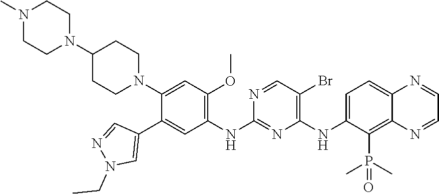 [SMILES: CCn1cc(-c2cc(Nc3ncc(Br)c(Nc4ccc5nccnc5c4P(C)(C)=O)n3)c(OC)cc2N2CCC(N3CCN(C)CC3)CC2)cn1]

### Step 1: Synthesis of Compound 19-1

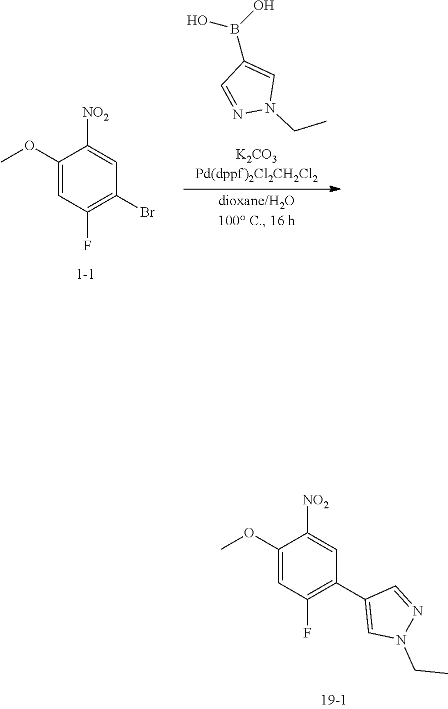

Prepare compound 19-1 as the method described for compound 1-2 using compound N-ethylpyrazol boronic acid instead of N-methylpyrazole boronic acid, MS: 266 [M+H]⁺

### Step 2: Synthesis of Compound 19-2

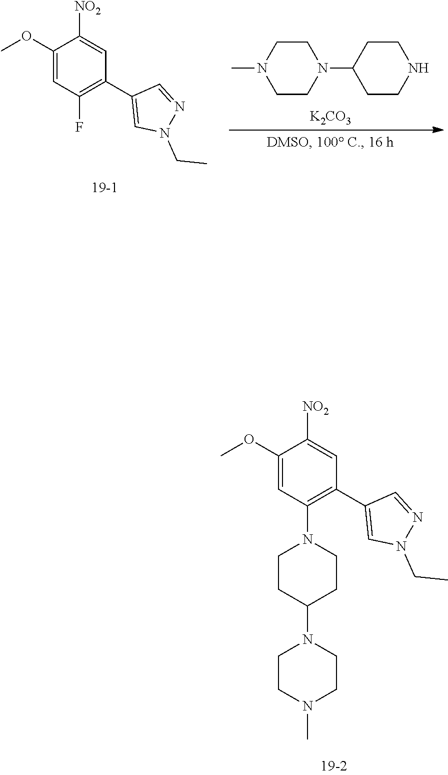

Prepare compound 19-2 as the method described for compound 1-3 using compound 19-1 instead of compound 1-2, MS: 429 [M+H]⁺

### Step 3: Synthesis of Compound 19-3

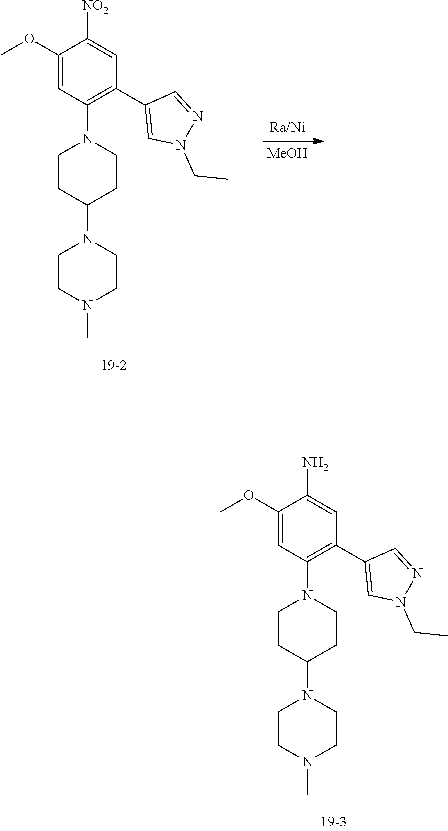 [SMILES: "MeOH" ; CCn1cc(-c2cc([N+](=O)[O-])c(OC)cc2N2CCC(N3CCN(C)CC3)CC2)cn1 ; CCn1cc(-c2cc(N)c(OC)cc2N2CCC(N3CCN(C)CC3)CC2)cn1]

Prepare compound 19-3 as the method described for compound 1-4 using compound 19-2 instead of compound 1-3, MS: 399 [M+H]⁺

### Step 4: Synthesis of Compound 19

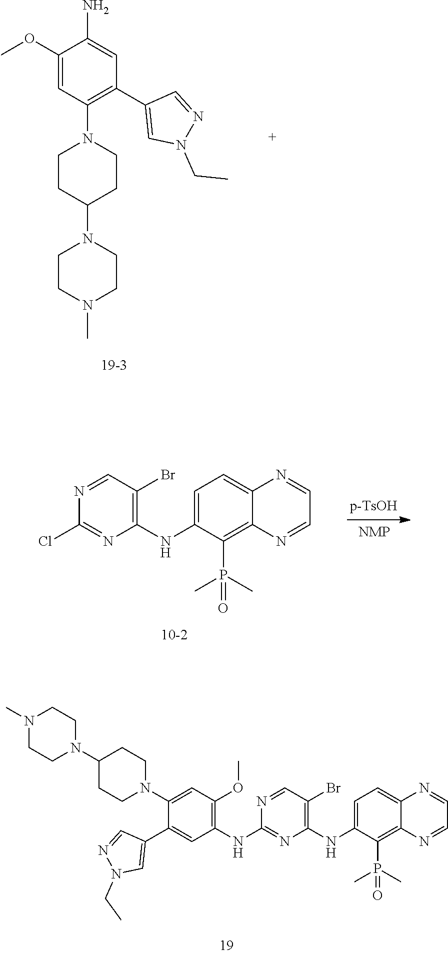

Prepare compound 19 as the method described for compound 1 using compound 19-3 instead of compound 1-4, compound 10-2 instead of compound 1-9- MS: 776 [M+H]⁺

### Example 20 Synthesis of Compound 20

### (6-((2-((4-([1,4′-Bipiperidine]-5-(1-ethyl-1H-pyrazol-4-yl)-2-methoxyphenyl) amino)-5-bromopyrimidine-4-yl)amino)-2,3-dimethylphenyl)dimethylphosphine oxide

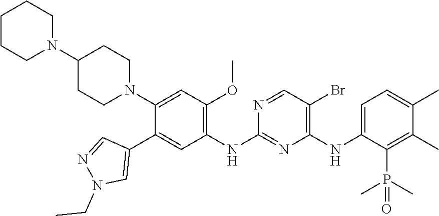 [SMILES: CCn1cc(-c2cc(Nc3ncc(Br)c(Nc4ccc(C)c(C)c4P(C)(C)=O)n3)c(OC)cc2N2CCC(N3CCCCC3)CC2)cn1]

### Step 1: Synthesis of Compound 20-1

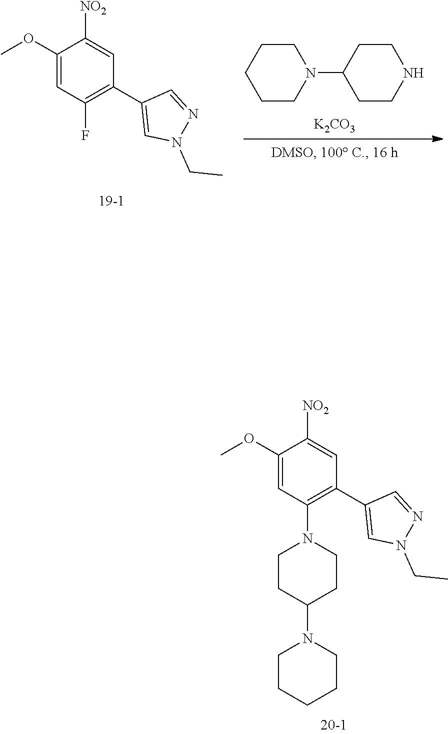

Prepare compound 20-1 as the method described for compound 19-2 using 4-piperidinopiperidine instead of 1-methyl-4(4-piperidinyl)piperazine, MS: 414 [M+H]⁺

### Step 2: Synthesis of Compound 20-2

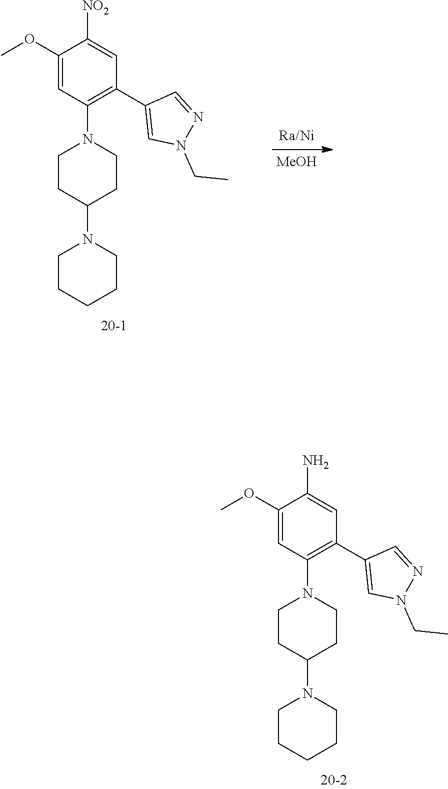 [SMILES: "MeOH" ; CCn1cc(-c2cc([N+](=O)[O-])c(OC)cc2N2CCC(N3CCCCC3)CC2)cn1 ; CCn1cc(-c2cc(N)c(OC)cc2N2CCC(N3CCCCC3)CC2)cn1]

Prepare compound 20-2 as the method described for compound 1-4 using compound 20-1 instead of compound 1-3, MS: 384 [M+H]⁺

### Step 3: Synthesis of Compound 20

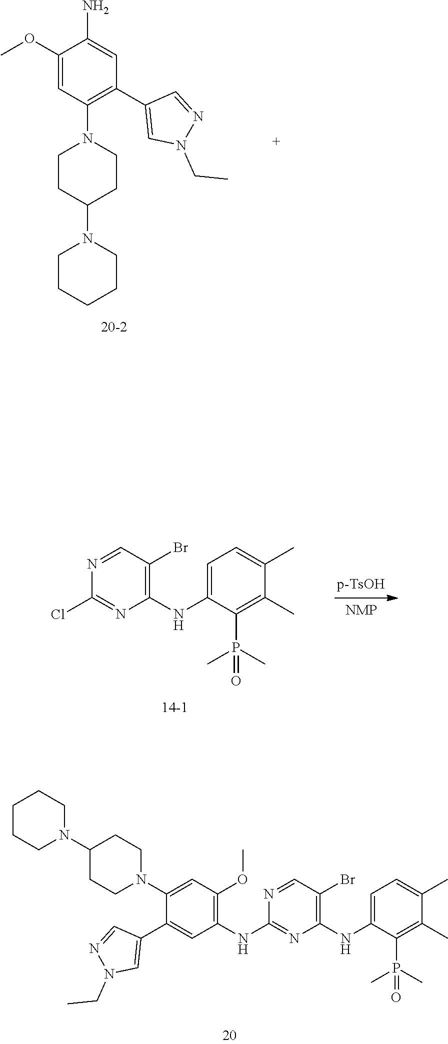

Prepare compound 20 as the method described for compound 1 using compound 20-2 instead of compound 1-4, compound 14-1 instead of compound 1-9, MS: 735 [M+H]⁺

### Example 21 Synthesis of Compound 21

### (6-((5-Chloro-2-((5-(1-methyl-1hydro-pyrazol-4-yl)-2-methoxy-4-morpholinophenyl) amino)pyrimidine-4-Amino)2,3-dimethylphenyl)-dimethylphosphine oxide

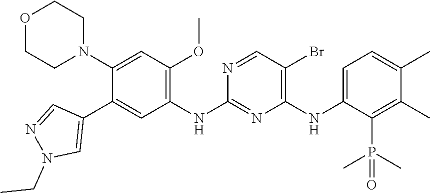 [SMILES: CCn1cc(-c2cc(Nc3ncc(Br)c(Nc4ccc(C)c(C)c4P(C)(C)=O)n3)c(OC)cc2N2CCOCC2)cn1]

### Step 1: Synthesis of Compound 21-1

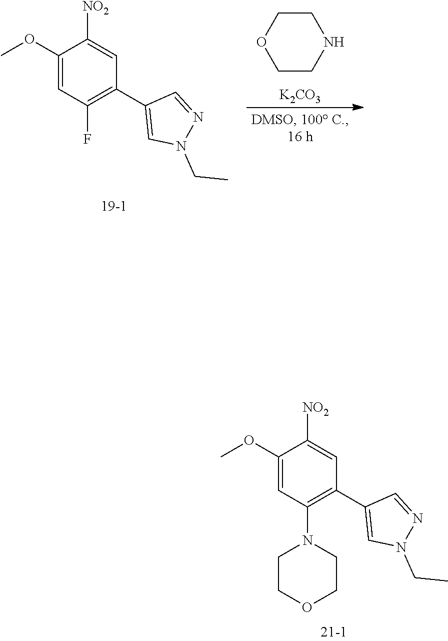

Preapare compound 21-1 as the method described for compound 19-2 using morpholine instead of compound 1-methyl-4-(4-piperidinyl)piperazine, MS:333 [M+H]⁺

### Step 2: Synthesis of Compound 21-2

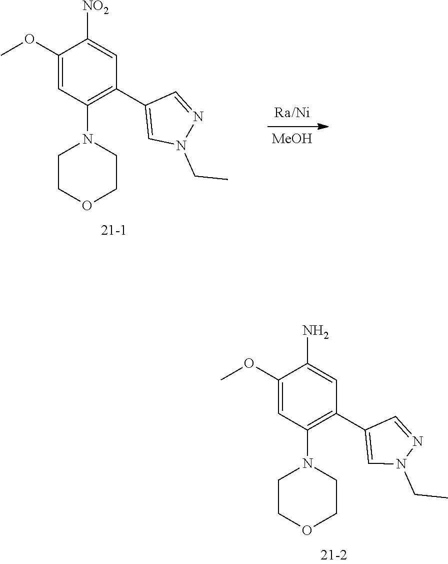 [SMILES: "MeOH" ; CCn1cc(-c2cc([N+](=O)[O-])c(OC)cc2N2CCOCC2)cn1 ; CCn1cc(-c2cc(N)c(OC)cc2N2CCOCC2)cn1]

Prepare compound 21-2 as the method described for compound 1-4 using compound 21-1 instead of compound 1-3, MS: 303 [M+H]⁺

### Step 3: Synthesis of Compound 21

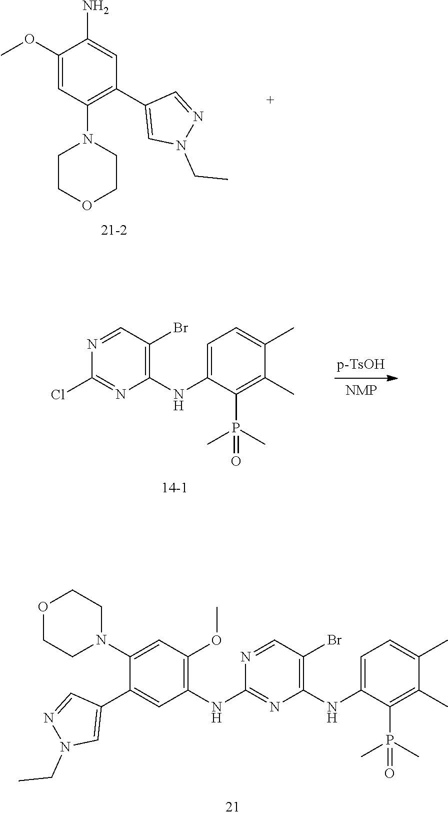

Prepare compound 21 as the method described for compound 1 using compound 21-1 instead of compound 1-4, compound 14-1 instead of compound 1-9. MS: 656 [M+H]⁺

### Example 22 Synthesis of Compound 22

### (6-((5-Bromo-2-((5-(1-ethyl-1h-pyrazol-4-yl)-2methoxy-4-(4-(4-methylpiperazine-1-yl) piperidin-1-yl)phenyl)amino)pyrimidine-4-yl)amino)-2,3-dimethylphenyl)dimethylphosphine oxide

### Synthesis of Compound 22

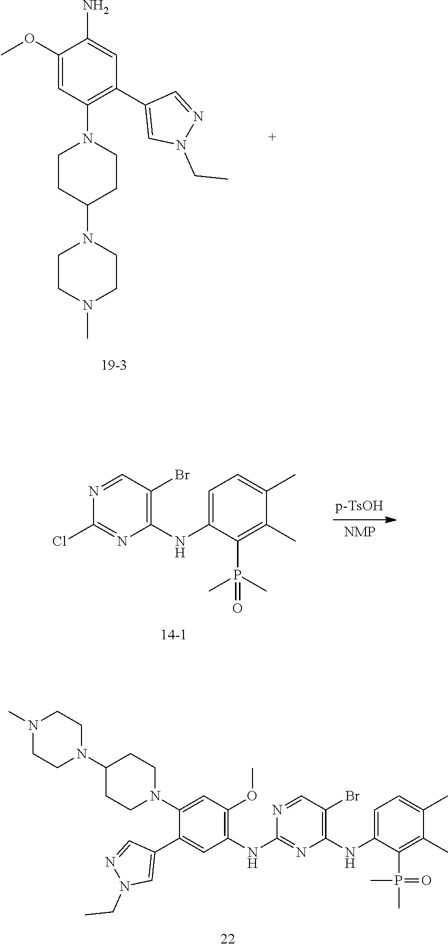

Prepare compound 22 as the method described for compound 1 using compound 19-3 instead of compound 1-4, compound 14-1 instead of compound 1-9, MS: 750 [M+H]⁺

### Example 23 Synthesis of Compound 23

### (6-(2-(5-((1-Ethyl-1hydro-pyrazol-4-yl)-2-methoxy-4-morpholinophenyl) amino)pyrimidine-4-amino)-2,3dimethylphenyl) dimethylphosphine oxide

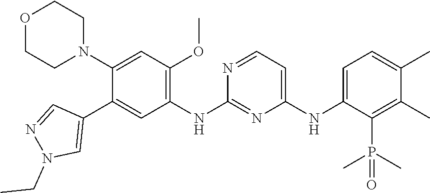 [SMILES: CCn1cc(-c2cc(Nc3nccc(Nc4ccc(C)c(C)c4P(C)(C)=O)n3)c(OC)cc2N2CCOCC2)cn1]

### Step 1: Synthesis of Compound 23-1

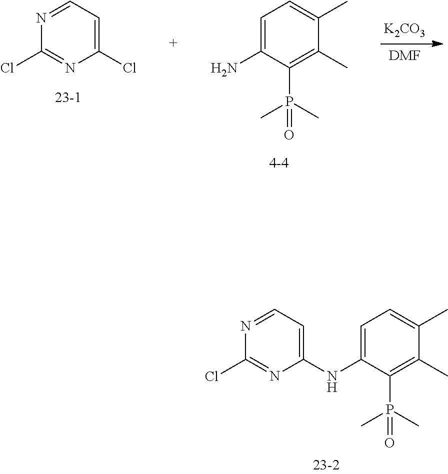

Prepare compound 23-2 as the method described for compound 4-5 using compound 23-1 instead of compound 1-8, MS: 310 [M+H]⁺

### Step 2: Synthesis of Compound 23

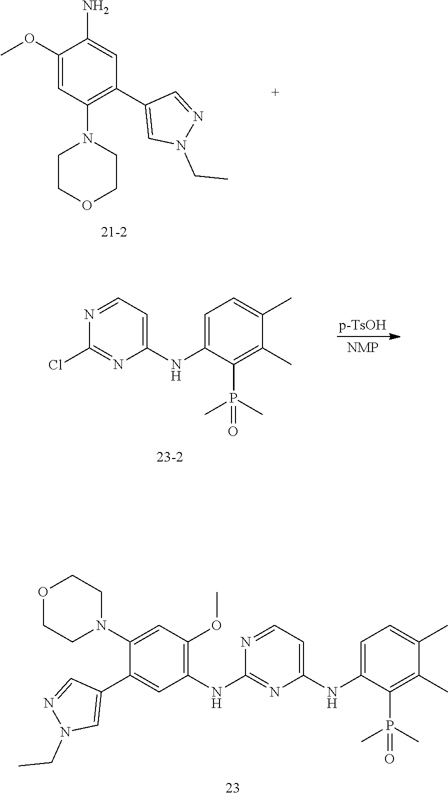 [SMILES: CCn1cc(-c2cc(N)c(OC)cc2N2CCOCC2)cn1 ; Cc1ccc(Nc2ccnc(Cl)n2)c(P(C)(C)=O)c1C ; CCn1cc(-c2cc(Nc3nccc(Nc4ccc(C)c(C)c4P(C)(C)=O)n3)c(OC)cc2N2CCOCC2)cn1]

Prepare compound 23 as the method described for compound 24 using compound 23-2 instead of compound 14-1, MS: 576 [M+H]⁺

### Example 24: Synthesis of Compound 24

### (6-((5-Bromo-2-((5-(1-ethyl-1H-pyrazol-4-yl)-2-methoxy-4-morpholinophenyl) amino)pyrimidine-4-Amino)-2-cyclopropylquinolin-5-yl)dimethylphosphine oxide

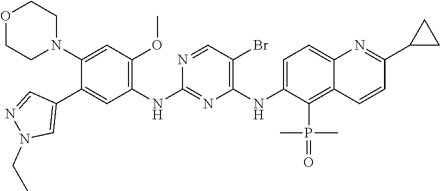 [SMILES: CCn1cc(-c2cc(Nc3ncc(Br)c(Nc4ccc5nc(C6CC6)ccc5c4P(C)(C)=O)n3)c(OC)cc2N2CCOCC2)cn1]

### Step 1: Synthesis of Compound 24-2

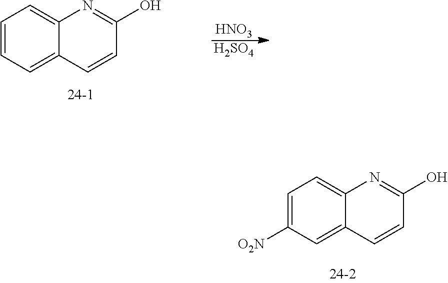

In a 100 ml single-neck flask, dissolve compound 24-1 (25 g, 172.23 mmol) in concentrated sulfuric acid (100 mL), and then add concentrated nitric acid (16.28 g, 258.34 mmol) dropwise into it at 0° C., and then stir for 2 h at room temperature. Complete reaction of the raw materials under TLC monitoring. Pour the reaction solution slowly into 2 L of ice water to quench, precipitate a light yellow solid, stir for 1 h, and be filtered; the filter cake was rinsed with 1 L of water, then collect the filter cake, and dried it to obtain compound 24-2 (24.5 g, 128.84 mmol), yield rate: 74.81%), MS: 191 [M+H]⁺

### Step 2: Synthesis of Compound 24-3

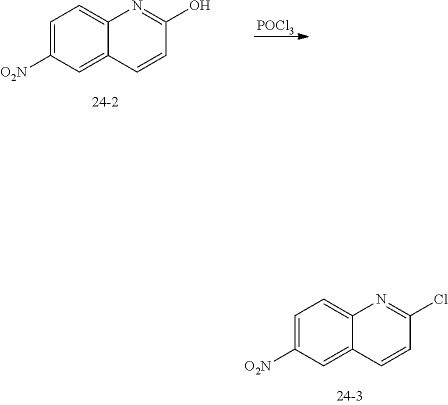

In a 500 ml single-neck flask, dissolve compound 24-2 (24.5 g, 128.84 mmol) in phosphine oxychloride (200 mL), heat to 100° C. and stir overnight. LCMS monitored the complete reaction of the raw materials. The reaction solution was cooled down to room temperature, and concentrated; the residue was poured into 1 L of ice water, stirred for 0.5 h, filtered, and the filter cake was rinsed with 1 L of water. The filter cake was collected and dried to obtain compound 24-3 (25 g, 119.85 mmol, yield rate: 93.02%).

### Step 3: Synthesis of Compound 24-4

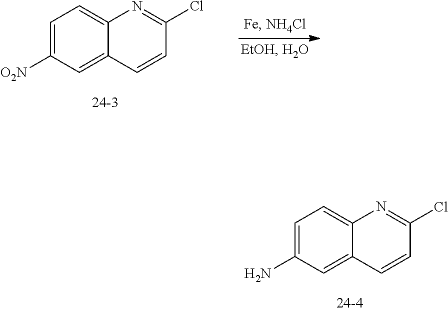 [SMILES: O=[N+]([O-])c1ccc2nc(Cl)ccc2c1 ; Nc1ccc2nc(Cl)ccc2c1]

In a 500 ml single-neck flask, dissolve compound 24-3 (25 g, 119.85 mmol) in 150 mL ethanol, add 30 mL H₂O, and then add iron powder (33.47 g, 599.23 mmol), ammonium chloride (32.05 g, 599.23 mmol), heat the reaction solution to 90° C. and stir for 3 h. The reaction solution was cooled down to room temperature, and filtered with celite; the filter cake was rinsed with ethanol several times, and the filtrate was collected and concentrated. The residue was purified by Flash silica gel column (A: DCM, B: MeOH; methanol from 0-5%, running for 20 min) to obtain compound 24-4 (16.9 g, 94.62 mmol, yield rate: 78.95%). MS: 179 [M+H]⁺

### Step 4: Synthesis of Compound 24-5

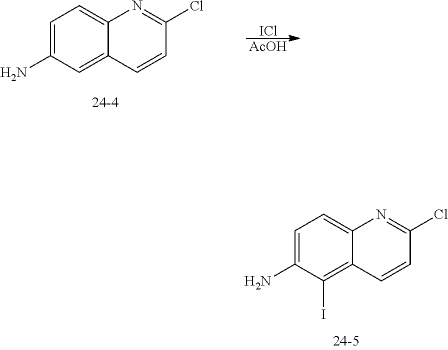 [SMILES: "AcOH" ; Nc1ccc2nc(Cl)ccc2c1 ; "ICl" ; Nc1ccc2nc(Cl)ccc2c1I]

In a 250 ml single-neck flask, dissolve compound 24-4 (16.9 g, 94.62 mmol) in glacial acetic acid (320 mL), add a solution of iodine chloride (18.43 g, 113.54 mmol) dropwise in acetic acid (80 mL) at room temperature, then stir at room temperature for 2 h. Complete reaction of the raw materials under LCMS monitoring. Adding 500 ml of n-hexane to the reaction liquid to dilute, and solids were precipitated out and then filtered: rinse the filter cake with n-hexane, and drain. The filter cake was dissolved in a mixed solvent of DCM:MeOH=10:1, washed with saturated sodium carbonate solution twice, saturated sodium thiosulfate solution twice, saturated sodium chloride washed once, dried, filtered, and concentrated. The residue was purified by Flash silica gel column (A:DCM, B:MeOH; MeOH from 0-5%, 20 min) to obtain compound 24-5 (22.23 g, 73.00 mmol, yield rate: 77.16%). MS: 305 [M+H]⁺

### Step 5: Synthesis of Compound 24-6

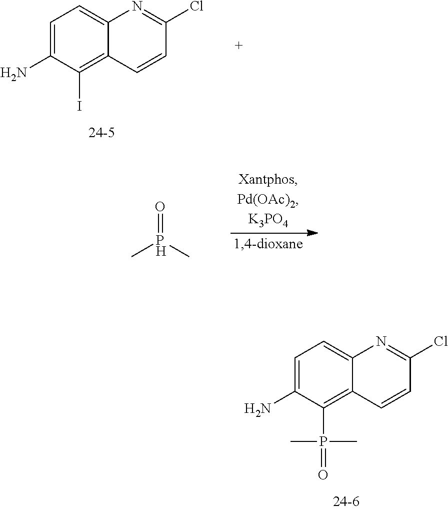

In a 250 ml single-neck flask, dissolve compound 24-5 (10.00 g, 32.84 mmol), dimethyl phosphine oxide (2.69 g, 34.48 mmol), Xantphos (3.80 g, 6.57 mmol), palladium acetate (737.27 mg, 3.28 mmol) and anhydrous potassium phosphate (13.94 g, 65.68 mmol) into 1,4-dioxane (100 mL), treat the solution with nitrogen displacement 3 times, heat it to 100° C. and be stifled overnight. The temperature was lowered, the reaction solution was filtered, and the filtrate was concentrated; the residue was purified by Flash silica gel column, first with petroleum ether/ethyl acetate (ethyl acetate from 0-50%, running for 10 mins), and then with dichloromethartelmethanol (methanol 0-6%, 20 min) to obtain compound 24-6 (6.18 g, 24.27 mmol, yield rate: 73.90%). MS: 255 [M+H]⁺

### Step 6: Synthesis of Compound 24-7

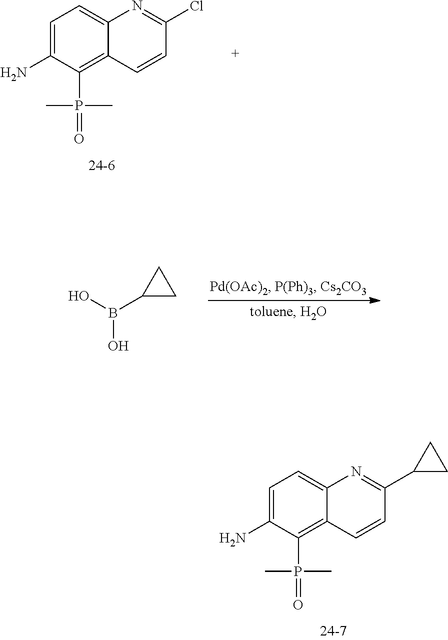 [SMILES: CP(C)(=O)c1c(N)ccc2nc(Cl)ccc12 ; OB(O)C1CC1 ; CP(C)(=O)c1c(N)ccc2nc(C3CC3)ccc12]

Add compound 24-6 (4 g, 15.71 mmol), cyclopropylboronic acid (5.40 g, 62.83 mmol), palladium acetate (352.65 mg, 1.57 mmol), triphenylphosphine (0.82 g, 3.14 mmol), Cs₂CO₃ (15.35 g, 47.12 mmol) into the mixed solvent of toluene (60 mL) and H₂O (10 mL), heat to 100° C. under nitrogen protection, and stir for 12 h. Complete reaction under LCMS monitoring, and cool down to room temperature. Add 40 mL of water, separate the layers, take the organic phase, extract the aqueous phase with ethyl acetate (3×30 mL), combine the organic phases, dry with anhydrous sodium sulfate, filter, concentrate, and purify by column chromatography (dichloromethane: methanol=15:1). The yellow solid, compound 24-7 (2.2 g, 8.45 mmol, yield rate: 53.81%) was obtained. MS: 261 [M+H]⁺

### Step 7: Synthesis of Compound 24-8

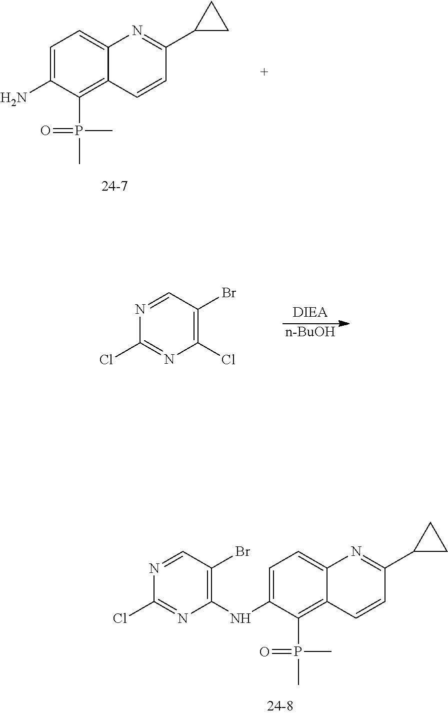

Add compound 24-7 (2.2 g, 8.45 mmol), 5-bromo-2,4-dichloropyrimidine (3.85 g, 16.91 mmol), DIEA (3.28 g, 25.36 mmol, 4.42 mL), n-BuOH (40 mL) successively into the reaction flask, heat to 120° C. and stir for 10 h. LCMS monitored the completion of the reaction. Cool down the reaction to room temperature; filter the solution, and dry the filter cake to obtain a pale yellow solid, the compound 24-8 (2.4 g, 5.31 mmol, yield rate: 62.86%). MS: 451 [M+H]⁺

### Step 8: Synthesis of Compound 24

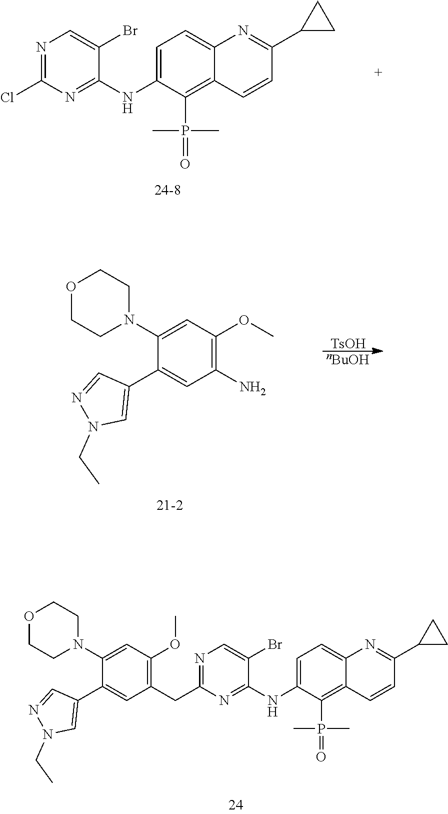

Prepare compound 24 as the method described for compound 21 using compound 24-8 instead of compound 14-1. MS: 717 [M+H]⁺

### Example 25 Synthesis of Compound 25

### (6-((5-Bromo-2-((4-(4-cyclopentylpiperazine-1yl)-2-methoxy-5-(1-methyl-1H-pyrozole-4-yl) phenyl)amino)pyrimidin-4-yl)amino)quinoxalin-5-yl) dimethylphosphine oxide

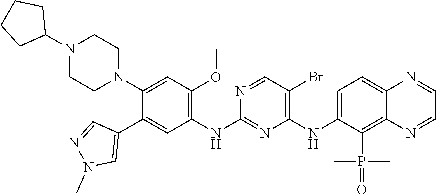 [SMILES: COc1cc(N2CCN(C3CCCC3)CC2)c(-c2cnn(C)c2)cc1Nc1ncc(Br)c(Nc2ccc3nccnc3c2P(C)(C)=O)n1]

### Step 1: Synthesis of Compound 25-1

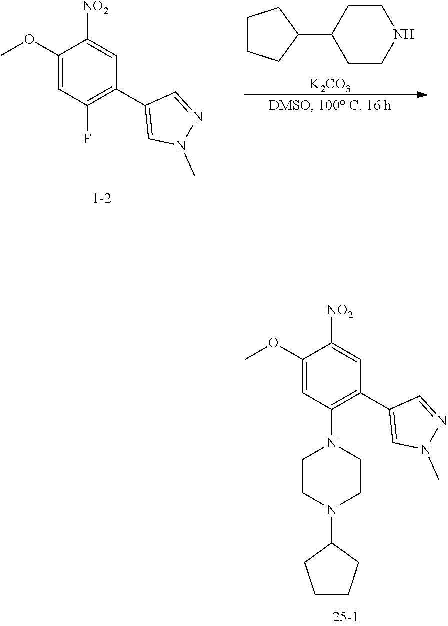

Prepare compound 6-1 as the method described for compound 6-1 using compound 4-cyclopentylpiperidine instead of compound 4-pyrrolidine-1-piperidine. MS: 386 [M+H]⁺

### Step 2: Synthesis of Compound 25-2

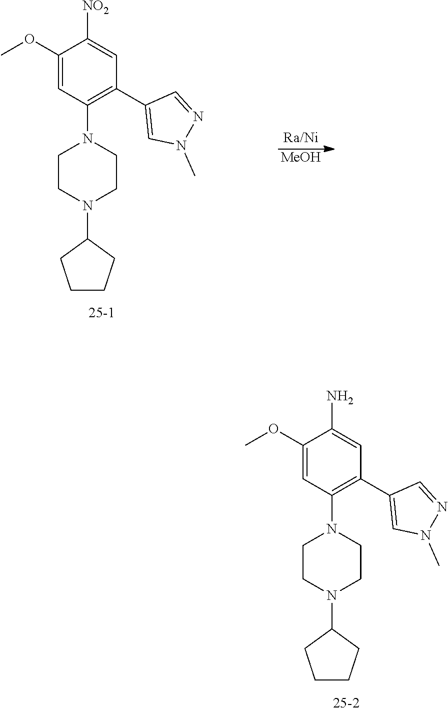 [SMILES: "MeOH" ; COc1cc(N2CCN(C3CCCC3)CC2)c(-c2cnn(C)c2)cc1[N+](=O)[O-] ; COc1cc(N2CCN(C3CCCC3)CC2)c(-c2cnn(C)c2)cc1N]

Prepare compound 25-2 as the method described for compound 6-2 using compound 25-2 instead of compound 6-1. MS: 356 [M+H]⁺

### Step 3: Synthesis of Compound 25

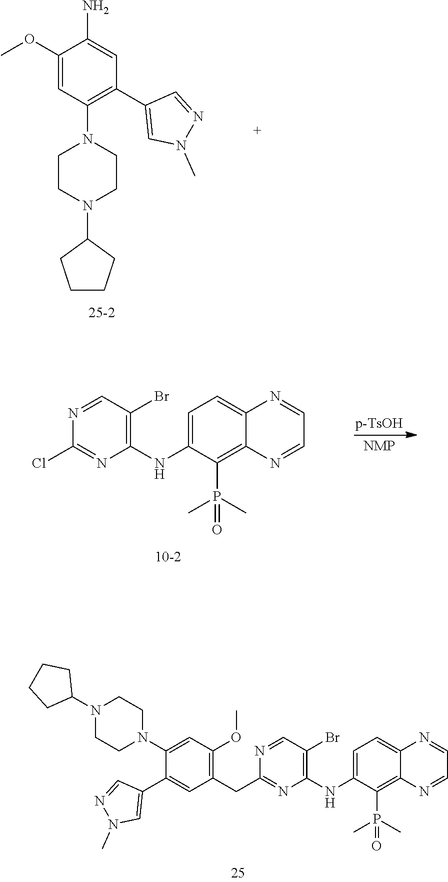 [SMILES: COc1cc(N2CCN(C3CCCC3)CC2)c(-c2cnn(C)c2)cc1N ; CP(C)(=O)c1c(Nc2nc(Cl)ncc2Br)ccc2nccnc12 ; COc1cc(N2CCN(C3CCCC3)CC2)c(-c2cnn(C)c2)cc1Cc1ncc(Br)c(Nc2ccc3nccnc3c2P(C)(C)=O)n1]

Prepare compound 25 as the method described for compound 6 using compound 25-2 instead of compound 6-2, compound 10-2 instead of compound 4-5. MS: 731 [M+H]⁺

### Example 26 Synthesis of Compound 26

### (6-((5-Bromo-2-((4-(4-cyclopentylpiperazine-1-yl)-2-methoxy-5-(1-methyl-1H-pyrazole-4-yl) phenyl)amino)pyrimidin-4-yl)amino)-2,3-dimethylphenyl)dimethylphoshine oxide;

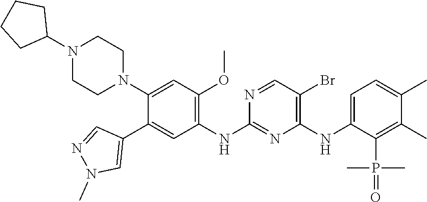 [SMILES: COc1cc(N2CCN(C3CCCC3)CC2)c(-c2cnn(C)c2)cc1Nc1ncc(Br)c(Nc2ccc(C)c(C)c2P(C)(C)=O)n1]

### Synthesis of Compound 26

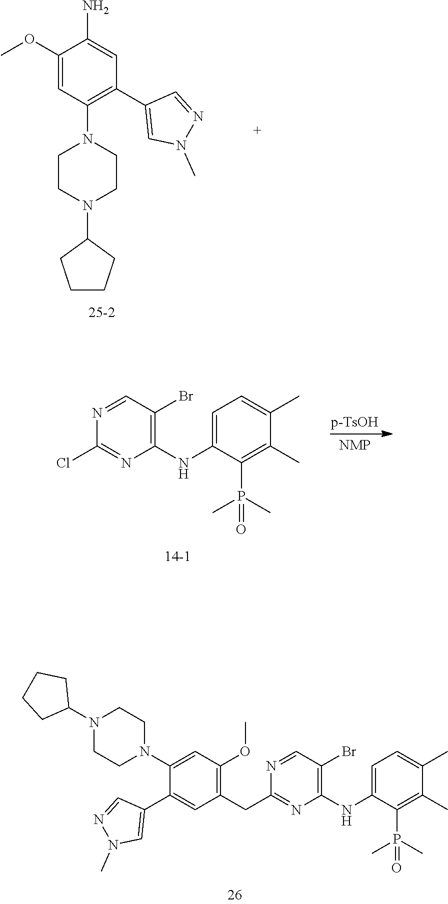 [SMILES: COc1cc(N2CCN(C3CCCC3)CC2)c(-c2cnn(C)c2)cc1N ; Cc1ccc(Nc2nc(Cl)ncc2Br)c(P(C)(C)=O)c1C ; COc1cc(N2CCN(C3CCCC3)CC2)c(-c2cnn(C)c2)cc1Cc1ncc(Br)c(Nc2ccc(C)c(C)c2P(C)(C)=O)n1]

Prepare compound 26 as the method described for compound 14 using compound 25-2 instead of compound 1-4. MS: 707 [M+H]⁺

### Example 27 Synthesis of Compound 27

### (6-((5-Bromo-2-((4-(4-cyclopentylpiperazine-1-yl)-2-methoxy-5-(1-methyl-1H-pyrazole-4-Yl) phenyl)amino)pyrimidin-4-yl)amino)-2-cyclopropylquinolin-5-yl)dimethylphosphine oxide;

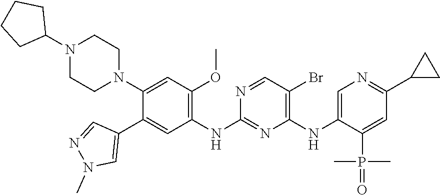 [SMILES: COc1cc(N2CCN(C3CCCC3)CC2)c(-c2cnn(C)c2)cc1Nc1ncc(Br)c(Nc2cnc(C3CC3)cc2P(C)(C)=O)n1]

### Synthesis of Compound 27

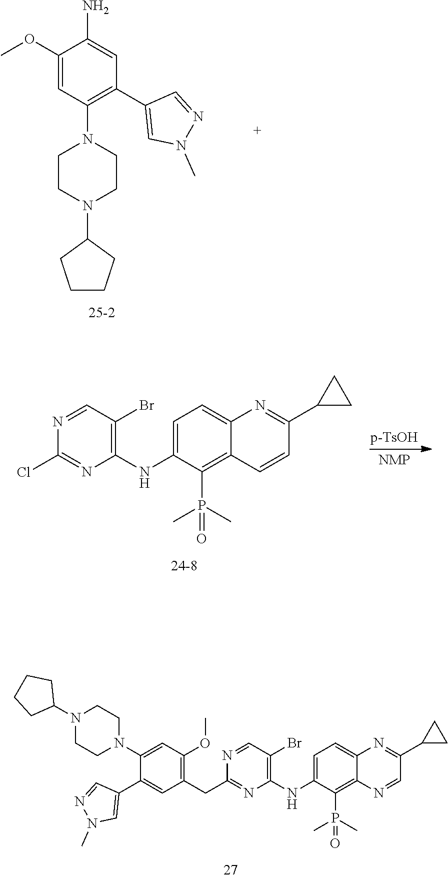 [SMILES: COc1cc(N2CCN(C3CCCC3)CC2)c(-c2cnn(C)c2)cc1N ; CP(C)(=O)c1c(Nc2nc(Cl)ncc2Br)ccc2nc(C3CC3)ccc12 ; COc1cc(N2CCN(C3CCCC3)CC2)c(-c2cnn(C)c2)cc1Cc1ncc(Br)c(Nc2ccc3nc(C4CC4)cnc3c2P(C)(C)=O)n1]

Prepare compound 27 as the method described for compound 25 using compound 24-8 instead of compound 25-5. MS: 770[M+H]⁺

### Example 28 Synthesis of Compound 28

Compound 28 (6-((5-Chloro-2-((2-methoxy-5-(1-methyl-1H-pyrazol-4-yl)-4-(4-(4-methylpiperazine-1-yl)piperidine-1-yl) phenyl)amino)pyrimidin-4-yl)amino)-2-cyclopropylquinolin-5-yl)dimethylphosphine oxide)

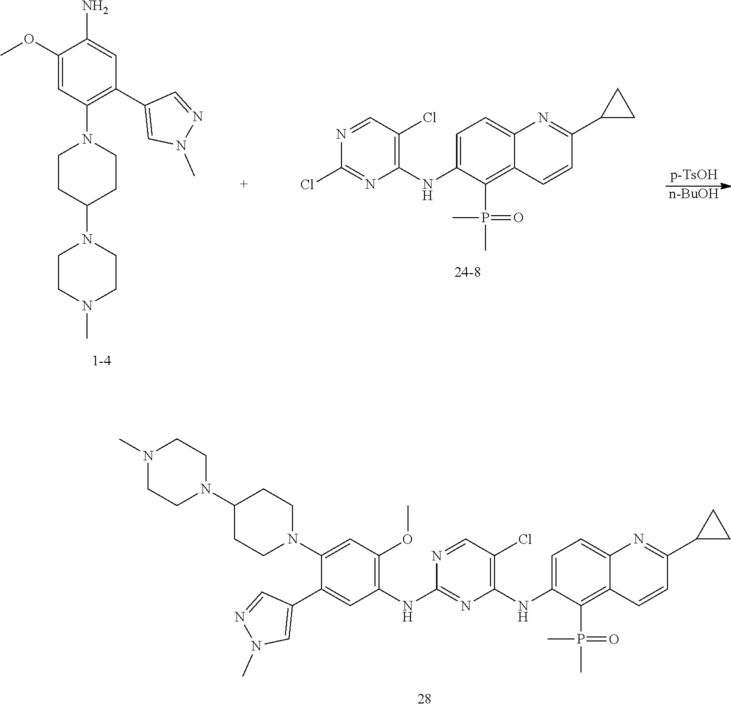

Add 1-4 (100 mg) and 24-8 (105 mg) into a 10 mL single-neck bottle, dissolve them in n-BuOH (4 mL), and then add p-TsOH (112 mg) into the bottle, keep them for chemical reaction at 110° C. for 4 h, complete reaction under LCMS and cool down to room temperature, followed by adding 10 mL dichloroinethane to dilute, washing with aqueous sodium bicarbonate and then with water, concentration and FLASH depuration (CH₃CN:H₂O=5%˜50%) to get the 92 mg product. MS: 755 [M+H]⁺

### Comparative Compound A

Prepare comparative compound A by the method described in Example 40 according WO2019015655.

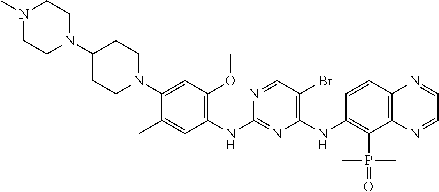 [SMILES: COc1cc(N2CCC(N3CCN(C)CC3)CC2)c(C)cc1Nc1ncc(Br)c(Nc2ccc3nccnc3c2P(C)(C)=O)n1]

### PHARMACOLOGICAL TESTING

### Test 1 EGFR A19de1 IT790M/C797S kinase test

Mobility variation analysis was performed to determine the affinity of the compound for EGFRΔ19del/T790M/C797S. The enzymatic reaction scheme is as follows:

1. Prepare 1* kinase buffer as follows:

| | Final |
|---|---|
| 1* kinase buffer | concentration |
| HEPES PH7.5 (mM) | 50 |
| Brij-35 | 0.0150% |
| DTT (mM) | 2 |
| Mgcl₂, Mncl₂ (mM) | 10 |

2. Preparation of compound concentration gradient: The test compound was tested with an initial concentration of 3000 nM or 100 nM, diluted in a 384 source plate to a 100% DMSO solution with 100-fold final concentration, and the compound is 3-fold diluted with Precision to 10 concentrations. A dispenser Echo 550 was used to transfer 250 nL 100-fold final concentration of the compound to the target plate OptiPlate-384F.

3. Prepare 2.5-fold final concentration of kinase solution with 1×Kinase buffer;

4. Add 10 μL of 2.5-fold final concentration of kinase solution to the compound well and the positive control well; add 10 μL of 1×Kinase buffer to the negative control well.

5. Centrifuge at 1000 rpm for 30 seconds, shake the reaction plate for well mixation, and then incubate at room temperature for 10 minutes.

6. Prepare a mixture of ATP and Kinase substrate at 5/3-fold final concentration with 1×Kinase buffer.

7. Add 15 μL mixture of 5/3-fold final concentration of ATP and substrate to start reaction.

8. Centrifuge the 384-well plate at 1000 rpm for 30 seconds, shake it well and then incubate it at room temperature for corresponding time.

9. Add 30 μL of stop detection solution to stop the kinase reaction, centrifuge at 1000 rpm for 30 seconds, shake it well.

10. Read conversion rate with Caliper EZ Reader.

11. Calculation formula

% Inhibition=((Conversion%_max−Conversion%_sample)/(Conversion%_max−Conversion%_ₘᵢₙ))*100

Wherein: Conversion%_sample is the sample conversion rate reading; Conversion%_min: Mean value of negative control wells, representing the conversion rate reading of wells without enzymatic activity; Conversion%_max: Mean value of positive control wells, representing the conversion rate reading of wells without compound inhibition.

The dose-effect curve was fitted with concentration log value as X-axis and percentage inhibition rate as Y-axis. Log (inhibitor) vs. Response—Variable Slope of GraphPad Prism 5 was used to fit the dose-effect curve to obtain IC50 value of enzymatic activity for each compound.

The calculation formula is as Y=Bottom (Top−Bottom)/(1+10\hat{ }((LogIC₅₀−X)*HillSlope)).

The results are expressed as IC50 values, as shown in Table 1. As illustrated in examples, IC₅₀ value is within the following range for the compound of the present invention: “A” represents “IC₅₀≤1nM”; “B” represents “1 nM<IC₅₀≤100nM”.

TABLE 1

| | EGFR |
|---|---|
| | Δ19del/T790M/C797S |
| No. | IC₅₀ (nM) |
| 1 | 0.3 |
| 2 | 0.2 |
| 3 | 0.3 |
| 4 | A |
| 5 | A |
| 6 | A |
| 7 | B |
| 8 | A |
| 9 | 0.3 |
| 10 | A |
| 11 | A |
| 12 | A |
| 13 | A |
| 14 | A |
| 15 | A |
| 16 | A |
| 17 | 0.3 |
| 18 | 0.2 |
| 19 | 0.3 |
| 20 | 0.2 |
| 21 | 0.3 |
| 22 | 4.2 |
| 23 | 1.4 |
| 24 | 17 |
| 25 | 0.3 |
| 26 | 0.2 |
| 27 | 1.3 |
| 28 | 0.31 |

### Test 2 Ba/F3-Δ19del/T790M/C797S and Ba/F3-L858R/T790M/C797S Cell Proliferation Assay

1. Cell Culture

Cell line: Ba/F3 cells with stable overexpression of Δ19del/T790M/C797S or L858R/T790M/C797S mutant genes, named Ba/F3-Δ19del/T790M/C797S and Ba/F3-L858R/T790M/C797S, and A431 EGFR wild-type cell line.

A. Culture Medium

RPMI 1640 and 10% FBS and 1% PS; _EM, 10% PBS and 1% PS

B. Cell Recovery

a) Preheat the medium in a 37° C. water bath;

b) Remove the cryotube from the liquid nitrogen tank, quickly put it in a 37° C. water bath for completely melting within 1 minute.

c) Transfer the cell suspension to a 15 mL centrifuge tube containing 8 mL of medium, and centrifuge at 1000 rpm for 5 minutes.

d) Discard the supernatant, resuspend the cells in 1 mL culture medium, transfer it to a 75 cm2 culture flask containing 15 mL of culture medium, and culture in a incubator with 5% CO₂ at 37° C.;

C. Cellpassage

a) Preheat the medium in a 37° C. water bath;

b) Collect the cells in a 15 mL centrifuge tube and centrifuge at 1000 rpm for 5 minutes. Discard the supernatant, count to make the cell density at 1×10⁴ cells/mL, and then place it in a incubator with 5% CO₂ at 37° C.

2. Compound Preparation

a) Dilute the test compound (20 mM stock solution) to 10 mM with 100% DMSO as the starting concentration, and then serially dilute 3 times with a “9+0” concentration in 96-well dilution plate (Cat #P-05525, Labcyte);

b) Dilute compound solution thereof to 1:100 in medium to prepare 10-fold working solution;

3. Cell Plate Culture

a) Centrifuge the growth cells in logarithmic phase at 1000 rpm for 5 minutes, resuspend the cells in culture medium, and then count the cells;

b) Inoculate the cells into a 96-well cell culture plate with a density of 2000 cells/well;

4. Compound Treatment

a) The compound prepared in Step 2 was added to a cell plate with 15 μL per well at final concentrations of 1000, 333, 111.1, 37, 12.3, 4.1, 1.4, 0.5, 0.2, and 0 nM. The final concentration of DMSO was 0.1%. The blank control well is with medium (0.1% DMSO).

b) Incubate cells in an incubatorother 72 hours;

5. Assay

a) Take out the 96-well cell culture plate and add 50 μl CTG reagent (CellTiter Glo kit, promega, Cat #G7573);

b) Shake the plate for 2 minutes and let cool at room temperature for 10 minutes,

c) Read luminous signal value with Perkin Elmer reader.

### Analysis of Experimental Data

The data were analyzed by GraphPad Prism 6.0 software to obtain the fitting curve of compound activity.

Fining compound IC₅₀ from nonlinear regression equation:

Y=Min+(Max−Min)/(1+10\hat{ }((LogIC₅₀−X)*slope));

X: Logarithm of compound concentration; Y: luminous signal

The result of cell proliferation assay is expressed by IC₅₀, as shown in Table 2. As illustrated in examples, IC₅₀ value is within the following range for the compound of the present invention: “A” represents “IC₅₀≤50 nM”; “B” represents “50 nM<IC₅₀<100 nM”.

TABLE 2

| | BaF3 | BaF3 | A431 |
|---|---|---|---|
| | L858R/T790M/C | Δ19del/T790M/C797S | EGFR WT |
| No. | 797S IC₅₀ (nM) | IC₅₀ (nM) | IC₅₀ (nM) |
| 1 | 2.1 | 2.1 | 112 |
| 2 | 18.3 | 6.5 | 109.9 |
| 3 | 60.4 | 60.4 | 571 |
| 4 | 14.9 | 10.2 | 191.9 |
| 5 | 6.5 | 3.0 | 114.1 |
| 6 | A | A | / |
| 7 | B | B | / |
| 8 | A | A | / |
| 9 | 16.8 | 8.0 | 251.8 |
| 10 | A | A | / |
| 11 | A | A | / |
| 12 | A | A | / |
| 13 | A | A | / |
| 14 | A | A | / |
| 15 | A | A | / |
| 16 | A | A | / |
| 17 | 3.8 | 3.5 | 120.5 |
| 18 | 8.2 | 6.7 | 111.5 |
| 19 | 1.3 | 1.0 | 135.3 |
| 20 | 0.4 | 0.7 | 151.7 |
| 21 | 3.6 | 0.7 | 465.0 |
| 22 | 31.1 | 24.6 | 3258 |
| 25 | 4.0 | 2.5 | 107 |
| 26 | 2.8 | 4.6 | 241 |
| 27 | 8.7 | 12.4 | 1174 |
| 28 | 6.6 | 1.0 | 201 |
| Comparative | 16.7 | 22 | 375 |
| compound A | | | |

Note:
″/” stands for “not tested”.

### Test 3 Liver Microsomal Clearance Test

1. Prepare 0.5 mg/mL liver microsome diluent containing K-butter and MgCl₂:

| | Concentration | Volume | Final |
|---|---|---|---|
| Reagent | of stock solution | added | concentration |
| MgCl₂ solution | 50 mM | 40 μL | 5 mM |
| K-buffer | 100 mM | 306 μL | 87 mM |
| Liver microsome | 20 mg/mL | 10 μL | 0.5 mg/mL |

2. Plate incubation:

2.1 Take 356 μL of the liver microsome diluent prepared above and add it to the preset wells of the incubation plate, the number of multiple wells n=1, mark it as the incubation plate.

2.2 Take 4 μL of the positive control compound Verapamil (50 μM) and the working solution of the test compound (200 μM) into the above incubation plate containing 356 μL of liver microsome diluent, and mix well (The dilution solvent from the stock solution to the working solution is a mixed solvent of acetonitrile: methanol: water=1:1:2, and the final concentration of DMSO is ≤0.5%),

2.3 Place the incubation plate in a 37° C. thermostatic oscillator (at shaking speed of 100 rpm) for pre-incubation with 10 min.

2.4 Add 40 μL of 10 mM NADPH (add 40 μL of K-buffer to the wells without NADPH) to each reaction well to start the reaction and start timing.

3. Sample collection and treatment:

3.1 After adding NADPH to start the reaction, immediately take out 50 μL of the sample and add it to the precipitation plate pre-added with 400 μL of stop solution, and shake on an oscillator at 600 rpm for 5 min at room temperature as a 0 min sample.

3.2 At the predetermined time points of 5 min, 15 min and 45 min, take out 50 μL of sample from the incubation plate and add it to the precipitation plate. The subsequent steps were the same as the treatment of 0 min sample. 3.3 After balancing, centrifuge above precipitation plate at 3200 rpm for 10 min at 4° C.

3.4 Take 50 μL of the supernatant after centrifugation and add it to pre-added 20% acetonitrile water at appropriate proportion, shake on an oscillator at 600 rpm for 2 min at room temperature, mix well, and put it into the LC-MS/MS sampler for sample injection.

### Analysis of Experimental Data

1. Respectively measure peak area of analyte in the sample and peak area of the internal standard at different time points, use the ratio of the peak area of the analyte to the peak area. of the internal standard at non-zero time point, and the ratio of peak area of the analyte in 0 min sample to the peak area of internal standard to calculate residual percentage of active drug; use the natural logarithmic value of the residual percentage as the ordinate, and the incubation time as the abscissa to plot, fit the straight line, and calculate the slope.

2. Calculation formula is as follows:

Elimination rate constant (K)=−slope;

Half-life period (T_{1/2})=0.693/K;

Intrinsic clearance (CLᵢₙₜ, in vitro)=K/Cₚᵣₒₜₑᵢₙ;

Note: Cₚᵣₒₜₑᵢₙ represents the concentration of liver microsomal protein in the final incubation system.

The CLᵢₙₜ data measured are shown in Table 3.

TABLE 3

| | Instrinsic clearance of liver microsomal | | | | |
|---|---|---|---|---|---|
| | protein, CLᵢₙₜ (μL/min/mg proteins) | | | | |
| No. | Human | Monkey | Dog | Rat | Mice |
| Comparative | 17.4 | 65.4 | 15.6 | 22 | 16.4 |
| compound 1 | | | | | |
| 1 | 2.4 | 38.8 | 1 | 4 | 6.6 |
| 5 | 15.4 | 43.2 | 25.8 | 0.8 | 11.2 |

### Test 4 hERG IC₅₀ Test

1. Thaw hERG kits (Tracer, E-4031 and Membranes) at room temperature;

2. Add 10 μL of positive compound E-4031 and 10 μL of test compound (10 mM) to 384 well plate (Corning, Art. No. 3658);

3. E-4031 is sequentially 5-fold diluted by pure water (10 concentration points in total), and the test compound is sequentially 5-fold diluted by DMSO (10 concentration points in total).

4. Take 1 μL of all gradient concentrations and add them in parallel to the wells containing 24 μL Assay buffer, and dilute 25 times (take 3 μL of the maximum concentration of E-4031 and add it to 72 μL Assay buffer).

5. Add 10 μL Membranes to another black 384-well plate (Coming, Art.4511).

6. Take 5 μL of 25-fold diluted working solution of positive and test compounds, and add it to above-mentioned plate with 10 μL Membranes, 2 copies at each concentration.

7. Negative control: Add 5 μL Assay buffer to the well with 10 μL Membranes, 8 copies in parallel;

8. Positive control: Add 5 μL E-4031 (120 μM) to the well with 10 μL Membranes, 8 copies in parallel;

9. Dilute a certain amount of Tracer solution at the ratio of 1:61,5, and then add 5 μL to all the wells pre-added positive compound, test compound, negative control and positive control.

10. Before measuring the fluorescence polarization, cover the analysis plate to protect the reagents from light and evaporation, and incubate at room temperature (20-25° C.) for 2 hours.

11. Read fluorescence polarization in PerkinElmer EnVision® Multilabel after incubation;

12. Calculation formula:

Y=100/(1+10\hat{ }((LogIC₅₀−X)*HillSlope))

Wherein, X is concentration of test article (Log μM), Y is relative residual activity (%), HillSlope is slope, IC₅₀ and 95% confidence interval are calculated by Prism software. The assay results are shown in Table 4.

TABLE 4
IC₅₀ results of hERG protein to compound

| No. | IC₅₀ (μM) |
|---|---|
| Comparative | 13.5 |
| compound 1 | |
| 1 | >30 |

1. A compound of Formula I, or a stereoisomer, tautomer, deuterated compound, pharmaceutically acceptable salt, prodrug, chelate, non-covalent complex, or solvate thereof, 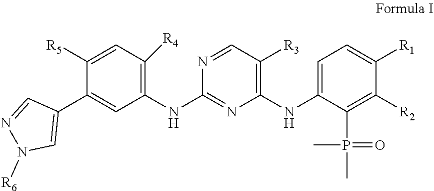
wherein,
R₁ and R₂ are independently selected from halogens, CN, NH₂, —C₁₋₆ alkyl, —C₁₋₆ alkoxy and —C₃₋₅ cycloalkyl, respectively; the NH₂, —C₁₋₆ alkyl, —C₁₋₆ alkoxy and —-C₃₋₅ cycloalkyl are optionally substituted with halogens and —C₁₋₄ alkyl; or
R₁ and R₂ together with the atoms to which they are attached form phenyl, —C₅₋₆ heteroaryl, —C₅₋₇ heterocyclyl or —C₅₋₆ cycloalkyl; the phenyl, —C₅₋₆ heteroaryl, —C₅₋₇ heterocyclyl or —C₅₋₆ cycloalkyl are optionally substituted with halogen, CN, NH₂, —C₁₋₆ alkyl;
R₃ is selected from hydrogen, halogen and —C₁₋₆ alkyl;
R₄ is selected from hydrogen, halogen, —C₁₋₆ alkyl, —C₁₋₆ alkoxy and —C₁₋₆ haloalkyl;
R₅ is selected from —C₅₋₆ heterocyclyl, 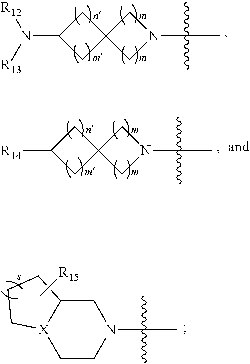
the —C₅₋₆ heterocyclyl is optionally substituted with —C₄₋₆ cycloalkyl, —C₄₋₆ heterocyclyl and —NR₇R₈;
R₆, R₇, R₈, R₁₂, R₁₃, R₁₄ and R₁₅ are independently selected from hydrogen, —C₁₋₆ alkyl and —C₁₋₆ halogenated alkyl, respectively;
X is selected from CH and N;
m, n, m′, n′, and s are independently selected from 1 and 2, respectively.

2. The compound of claim 1, or a stereoisomer, tautomer, deuterated compound, pharmaceutically acceptable salt, prodrug, chelate, non-covalent complex, or solvate thereof, wherein R₁ and R₂ are independently selected from —C₁₋₆ alkyl and —C₃₋₅ cycloalkyl.

3. The compound of claim 1, or a stereoisomer, tautomer, deuterated compound, pharmaceutically acceptable salt, prodrug, chelate, non-covalent complex, or solvate thereof, wherein R₁ and R₂ are independently selected from —CH₃ and

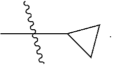 [SMILES: *C1CC1]

4. The compound of claim 1, or a stereoisomer, tautomer, deuterated compound, pharmaceutically acceptable salt, prodrug, chelate, non-covalent complex, or solvate thereof, wherein R₁ and R₂, together with the atoms to which they are attached form —C₅₋₆ heteroaryl; the —C₅₋₆ heteroaryl is optionally substituted with hydrogen, halogen, CN, —NH₂, —C₁₋₃ alkyl, and —C₃₋₅ cycloalkyl.

5. The compound of claim 1, or a stereoisomer, tautomer, deuterated compound, pharmaceutically acceptable salt, prodrug, chelate, non-covalent complex, or solvate thereof, wherein R₁ and R₂ together with the atoms to which they are attached form

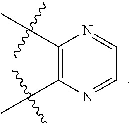 [SMILES: *c1nccnc1*]

6. The compound of claim 1, or a stereoisomer, tautomer, deuterated compound, pharmaceutically acceptable salt, prodrug, chelate, non-covalent complex, or solvate thereof, wherein R₁ and R₂ together with the atoms to which they are attached form

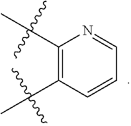 [SMILES: *c1cccnc1*]

7. The compound of claim 1, or a stereoisomer, tautomer, deuterated compound, pharmaceutically acceptable salt, prodrug, chelate, non-covalent complex, or solvate thereof, wherein R₃ is selected form halogen.

8. The compound of claim 1, or a stereoisomer, tautomer, deuterated compound, pharmaceutically acceptable salt, prodrug, chelate, non-covalent complex, or solvate thereof, wherein R₃ is selected form Cl or Br.

9. The compound of claim 1, or a stereoisomer, tautomer, deuterated compound, pharmaceutically acceptable salt, prodrug, chelate, non-covalent complex, or solvate thereof, wherein R₄ is selected from —C₁₋₆ alkoxy.

10. The compound of claim 1, or a stereoisomer, tautomer, deuterated compound, pharmaceutically acceptable salt, prodrug, chelate, non-covalent complex, or solvate thereof, wherein R₄ is selected form —O—CH₃.

11. The compound of claim 1, or a stereoisomer, tautomer, deuterated compound, pharmaceutically acceptable salt, prodrug, chelate, non-covalent complex, or solvate thereof, wherein R₅ is selected from —C₅₋₆ heterocyclyl.

12. The compound of claim 1, or a stereoisomer, tautomer, deuterated compound, pharmaceutically acceptable salt, prodrug, chelate, non-covalent complex, or solvate thereof, wherein R₅ is selected from

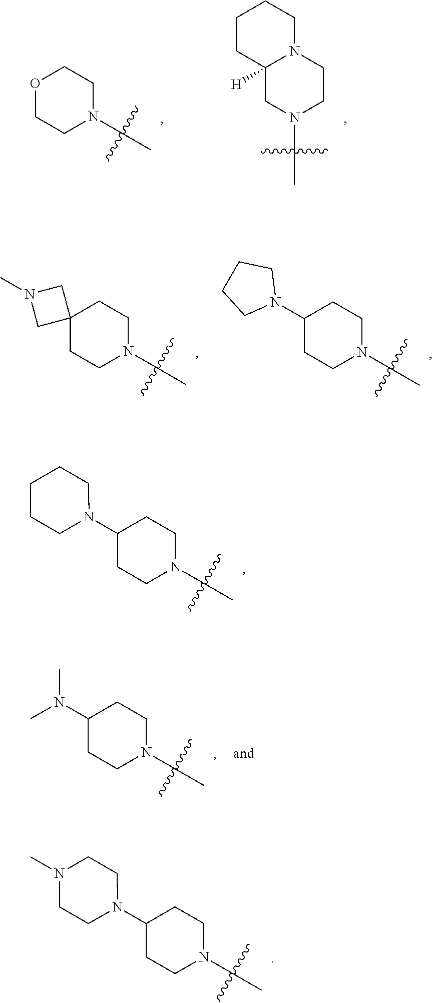

13. The compound of claim 1, or a stereoisomer, tautomer, deuterated compound, pharmaceutically acceptable salt, prodrug, chelate, non-covalent complex, or solvate thereof, wherein R₆ is selected from hydrogen, —C₁₋₃ alkyl and —C₁₋₃ halogenated alkyl.

14. The compound of claim 1, or a stereoisomer, tautomer, deuterated compound, pharmaceutically acceptable salt, prodrug, chelate, non-covalent complex, or solvate thereof, wherein R₆ is selected from H, CH₃, CH₂CH₃ and CHF₂.

15. The compound of claim 1, or a stereoisomer, tautomer, deuterated compound, pharmaceutically acceptable salt, prodrug, chelate, non-covalent complex, or solvate thereof, wherein the compound is:
1) (6-(5-Chloro-2-((2-methoxy-5-(1-methyl-1hydro-pyrazol-4-yl)-4-(4-(4-methylpiperazine-1-yl) piperidin-1-yl)phenyl)amino)pyrimidin-4-yl)amino)quinoxalin-5-yl)dimethylphosphine oxide;
2) (6-((5-Chloro-2-((4-(4-(dimethylamino)piperidin-1-yl)-2-methoxy-5-(1-methyl-1H-pyrazole-4-yl) phenyl)amino)pyrimidin-4-yl)amino)quinoxalin-5-yl)dimethylphosphine oxide;
3) (6-((5-Chloro-2-((4-(4-(dimethylamino)piperidin-1-yl)-2-methoxy-5-(1H-pyrazol-4-yl) benzene)amino)pyrimidin-4-yl)amino)quinoxalin-5-yl)dimethylphosphine oxide;
4) (6-((5-Chloro-2((4-(4-(dimethylamino)piperidin-1-yl)-2-methoxy-5-(1-methyl-1H-pyrazole-4-yl) phenyl)amino)pyrimidin-4-yl)amino)-2,3-dimethylphenyl)dimethylphosphine oxide;
5) (6-((2-((4-([1,4′-Dipiperidine]-1′-yl)-2-methoxy-5-(1-methyl-1H-pyrazole-4-yl) Phenyl)amino)-5-chloropyrimidin-4-yl)amino)quinoxalin-5-yl)dimethylphosphine oxide;
6) (6-((5-Chloro-2-((2-methoxy-5-(1-methyl-1H-pyrazol-4-yl)-4-(4-(pyrrol-1-yl) piperidine-1-yl)phenyl)amino)pyrimidin-4-yl)yl)-2,3-dimethylphenyl)dimethylphosphine oxide;
7) (6-((5-chloro-2-((2-methoxy-5-(1-methyl-1H-pyrazol-4-yl)-4-4-(2-methyl-2,7-diazaspiro[3.5]nonyl-7-yl)phenyl)amino)pyrimidin-4-yl)amino)-2,3-dimethylphenyl)dimethylphosphine oxide;
8) (R)-(6-((5-chloro-2-((2-methoxy-5-methyl-1H-pyrazol-4-yl)-4-(octahydro-2hydro-Pyridine [1,2-a]pyrazine-2-yl)phenyl)amino)pyrimidin-4-yl)amino)-2,3-dimethylphenyl)dimethylphosphine oxide;
9) (6-((5-chloro-2-(2-methoxy-5-(1-methyl-1hydro-pyrazole)-4-morpholinephenyl)amino) pyrimidine-4-amino)quinoxalin-5-dimethylphosphine oxide;
10) (6-((5-Bromo-2-((2-methoxy-5-(1-methyl-1H-pyrazol-4-yl)-4-(4-(4-methylpiperazine-1-yl) piperidin-1-yl)phenyl)amino)pyrimidin-4-yl)amino)quinoxalin-5-yl)dimethylphosphine oxide;
11) (6-((2((4-([1,4′-Bipiperidine]-1′-yl)-2-methoxy-5-(1-methyl-1H-pyrazole-4-yl) phenyl)amino)-5-bromopyrimidine-4-yl)amino)quinoxalin-5-yl)dimethylphosphine oxide;
12) (6-((5-Bromo-2-((2-methoxy-5-(1-methyl-1H-pyrazol-4-yl)-4-(4-(4-methylpiperazine-1-yl) piperidin-1-yl)phenyl)amino)pyrimidin-4-yl)amino)-3-cyclopropyl-2-methyl)dimethylphosphine oxide;
13) (6-((2-((4-([1,4′-Bipiperidine]-1′-yl)-2-methoxy-5-(1-methyl-1H-pyrazole-4-yl) phenyl)amino)-5-bromopyrimidine-4-yl)amino)-3-cyclopropyl-2-methylphenyl)dimethylphosphine oxide;
14) (6-((5-Bromo-2-((2-methoxy-5-(1-methyl-1h-pyrazol-4-yl)-4-(4-(4-methylpiperazine-1-yl) piperidin-1-yl)phenyl)amino)pyrimidin-4-yl)amino)-2,3-dimethylphenyl)dimethylphosphine oxide;
15) (6-((2-((4-([1,4′-Bipiperidine]-1′-yl)-2-methoxy-5-(1-methyl-1H-pyrazole-4-yl) phenyl)amino)-5-bromopyrimidin-4-yl)amino)-2,3-dimethylphenyl)dimethylphosphine oxide;
16) (6-((2-((4-([1,4′-Bipiperidine]-1′-yl)-2-methoxy-5-(1-methyl-1H-pyrazole-4-yl) phenyl)amino)-5-bromopyrimidine-4-yl)amino)-3-cyclopropyl-2-methylphenyl)dimethylphosphine oxide;
17) (6-((5-Bromo-2-((2-methoxy-5-(1-methyl-1h-pyrazol-4-yl)-4-(4-(4-methylpiperazine-1-yl) piperidin-1-yl)phenyl)amino)pyrimidin-4-yl)amino)-2,3-dimethylphenyl)dimethylphosphine oxide;
18) (R)-(6-((5-Chloro-2-((2-methoxy-5-([1-methyl-1hydro-pyrazol-4-yl]-4-(octahydro-2hydro-pyrazol[1,2-a]pyrazine-2-yl) phenyl)amino)pyrimidin-4-yl)amino)quinoxalin-5-yl)dimethylphosphine oxide;
19) (6-((5-Bromo-2-((5-(1-ethyl-1H-pyrazol-4-yl)-2-methoxy)-4-(4-(4-methylpiperazine-1-yl) piperidin-1-yl)phenyl)amino)pyrimidin-4-yl)amino)quinoxalin-5-yl)dimethylphosphine oxide;
20) (6-((2-((4-([1,4′-Bipiperidine]-1′-yl)-5-(1-ethyl-1H-pyrazol-4-yl)-2-methoxyphenyl) amino)-5-bromopyrimidine-4-yl)amino)-2,3-dimethylphenyl)dimethylphosphine oxide;
21) (6-((5-Chloro-2-((5-(1-methyl-1hydro-pyrazol-4-yl)-2-methoxy-4-morpholinophenyl) amino)pyrimidine-4-Amino)2,3-dimethylphenyl)-dimethylphosphine oxide;
22) (6-((5-Bromo-2-((5-(1-ethyl-1h-pyrazol-4-yl)-2-methoxy-4-(4-(4-methylpiperazine-1-yl) piperidin-1-yl)phenyl)amino)pyrimidine-4-yl)amino)-2,3-dimethylphenyl)dimethylphosphine oxide;
23) (6-(2-(5-((1-Ethyl-1hydro-pyrazol-4-yl)-2-methoxy-4-morpholinophenyl)amino) pyrimidine-4-amino)-2,3 dimethylphenyl)dimethylphosphine oxide;
24) (6-((5-Bromo-2-((5-(1-ethyl-1H-pyrazol-4-yl)-2-methoxy-4-morpholinophenyl) amino)pyrimidine-4-Amino)-2-cyclopropylquinolin-5-yl)dimethylphosphine oxide;
25) (6-((5-Bromo-2-((4-(4-cyclopentylpiperazine-1-yl)-2-methoxy-5-(1-methyl-1H-pyrazole-4-yl) phenyl)amino)pyrimidin-4-yl)amino)quinoxalin-5-yl)dimethylphosphine oxide;
26) (6-((5-Bromo-2-((4-(4-cyclopentylpiperazine-1-yl)-2-methoxy-5-(1-methyl-1H-pyrazole-4-yl) phenyl)amino)pyrimidin-4-yl)amino)-2,3-dimethylphenyl)dimethylphosphine oxide;
27) (6-((5-Bromo-2-((4-(4-cyclopentylpiperazine-1-yl)-2-methoxy-5-(1-methyl-1H-pyrazole-4-Yl) phenyl)amino)pyrimidin-4-yl)amino)-2-cyclopropylquinolin-5-yl)dimethylphosphine oxide; or
28) (6-((5-Chloro-2-((2-methoxy-5-(1-methyl-1H-pyrazol-4-yl)-4-(4-(4-methylpiperazine-1-yl) piperidine-1-yl)phenyl)amino)pyrimidin-4-yl)amino)-2-cyclopropylquinolin-5-yl)dimethylphosphine, oxide).

16. A pharmaceutical composition comprising a compound of claim 1, or a pharmaceutically acceptable salt or a stereoisomer thereof, and at least one pharmaceutically acceptable carrier or excipient.

17. A method of inhibiting various different forms of EGFR, including L858R, Δ19del, T790M, C797S and any combination of them, said method comprising administering to a patient a compound of claim 1, or a pharmaceutically acceptable salt thereof.

18. A method of treating EGFR-driven cancer, said method comprising administering to a patient in need thereof a therapeutically effective amount of a compound of any one of claims 1, or a pharmaceutically acceptable salt thereof.

19. The method of claim 18, wherein EGFR-driven cancer characterized by the presence of one or more mutations selected from, but not limited to: (i) C797S, (ii) L858R and C797S, (iii) C797S and T790M, (iv) L858R, T790M, and C797S, or (v) Δ19del , T790M and C797S.

20. The method of claim 18, wherein EGFR-driven cancers involve colon cancer, stomach cancer, thyroid cancer, lung cancer, leukemia, pancreatic cancer, melanoma, brain cancer, kidney cancer, prostate cancer, ovarian cancer, or breast cancer.

21. The method of claim 20, wherein lung cancer is non-small cell lung cancer caused by EGFR^{L858R/T790M/C797S} or EGFR^{Δ19del/T790M/C797S} mutant.

22. (canceled)

23. (canceled)

24. (canceled)

25. (canceled)
